(19) United States
(12) Patent Application Publication
(10) Pub. No.: US 2021/0048432 A1
(43) Pub. Date: Feb. 18, 2021

(54) DIRECT IMMUNOHISTOCHEMISTRY AND IMMUNOCYTOCHEMISTRY METHODS

(71) Applicant: NOVODIAX, INC., Hayward, CA (US)

(72) Inventors: Shuo CHEN, Milpitas, CA (US); Jin V. WU, Union City, CA (US); Jianfu WANG, Union City, CA (US)

(21) Appl. No.: 16/967,867

(22) PCT Filed: Feb. 8, 2019
(86) PCT No.: PCT/US2019/017315

§ 371 Date: Aug. 6, 2020

Related U.S. Application Data
(60) Provisional application No. 62/628,723, filed on Feb. 9, 2018.

(51) Int. Cl. G01N 33/547; C07K 16/46; C07K 19/00

(52) U.S. Cl. CPC G01N 33/547; C07K 2319/33; C07K 19/00; C07K 16/46

(57) ABSTRACT

The present disclosure provides methods, kits, and compositions for direct immunohistochemical (IHC) staining and direct immunocytochemistry (ICC) techniques, including applications to multiplex assays, chemically stained samples, and cytology samples.

### CROSS-REFERENCE TO RELATED APPLICATIONS

This application claims the priority benefit of U.S. Provisional Application No. 62/628,723, filed Feb. 9, 2018, the entire contents of which are hereby incorporated herein by reference.

### TECHNICAL FIELD

The present disclosure provides methods, kits, and compositions for direct immunohistochemical (IHC) staining and direct immunocytochemistry (ICC) techniques, including applications to multiplex assays, chemically stained samples, and cytology samples.

### BACKGROUND OF THE INVENTION

Immunohistochemical (IHC) staining of heterogeneous tissue samples is a reliable method of assessing the presence, or lack thereof, of target epitopes of target antigens, such as proteins. Generally, IHC techniques utilize an antibody to probe and visualize cellular antigens, e.g., target epitopes, in situ. However, due to the often diffuse distribution of target epitopes in tissues and cellular samples, signal amplification is necessary to visualize such cellular antigens. Known techniques for signal amplification include use of a secondary antibody that binds to the primary antibody specific to the cellular antigen and biotin-avidin systems. However, even with signal amplification techniques, IHC techniques may have limited application based on the concentration of a target epitope in a sample. For example, samples comprising target epitopes below the limit of detection (e.g., due to the diffuse nature of the target epitope or masking of the target epitopes by chemical staining) may not amenable to assessment by IHC techniques.

Use of IHC during intraoperative procedures may be a valuable source of information for improving patient treatments and outcomes. Intraoperative guidelines, such as those provided by the College of American Pathologists, typically recommend reporting pathology data to the surgeon within approximately 20 minutes. However, many IHC techniques may require 60 to 120 minutes to obtain results. The time required for performing an IHC technique may increase, e.g., when more than one target antigen is assessed and/or chemical staining is also applied to the sample.

All references cited herein, including patent applications and publications, are incorporated by reference in their entirety.

### BRIEF SUMMARY OF THE INVENTION

In one aspect, the present invention provides methods for detecting a plurality of target epitopes in a sample, the methods comprising: (a) a first antibody binding step comprising contacting the sample with a first polymeric-enzyme/antibody conjugate comprising a plurality of a first enzyme molecule and a first antibody recognizing a first target epitope in a condition suitable for forming a first complex comprising the first target epitope and the first polymeric-enzyme/antibody conjugate; (b) a second antibody binding step comprising contacting the sample with a second polymeric-enzyme/antibody conjugate comprising a plurality of a second enzyme molecule and a second antibody recognizing a second target epitope in a condition suitable for forming a second complex comprising the second target epitope and the second polymeric-enzyme/antibody conjugate; (c) a first antibody removal step comprising substantially removing from the sample the first polymeric-enzyme/antibody conjugate that does not form the first complex; (d) a second antibody removal step comprising substantially removing from the sample the second polymeric-enzyme/antibody conjugate that does not form the second complex; (e) a first enzyme substrate contact step comprising contacting the sample with a first enzyme substrate composition for the first enzyme molecule; and (f) a second enzyme substrate contact step comprising contacting the sample with a second enzyme substrate composition for the second enzyme molecule, thereby allowing for detection of the plurality of target epitopes in the sample.

In some embodiments, the first enzyme molecule and the second enzyme molecule are different. In some embodiments, the first antibody binding step and the second antibody binding step are performed simultaneously. In some embodiments, the first antibody removal step and the second antibody removal step are performed simultaneously. In some embodiments, the first enzyme substrate contact step and the second enzyme substrate contact step are performed simultaneously. In some embodiments, the first enzyme substrate contact step is performed prior to the second enzyme substrate contact step. In some embodiments, the first enzyme substrate contact step is performed after the second enzyme substrate contact step.

In some embodiments, the first antibody binding step is performed prior to the second antibody binding step. In some embodiments, the first antibody removal step and the first enzyme substrate contact step are performed prior to the second antibody binding step. In some embodiments, the methods further comprise a first antibody stripping step comprising dissociating the first antibody from the first target epitope, wherein the first antibody stripping step is performed prior to the second antibody binding step. In some embodiments, the methods further comprise a first enzyme inactivation step comprising inactivating the first enzyme molecule, wherein the first enzyme inactivation step is performed prior to the second antibody binding step.

In some embodiments, the first antibody binding step is performed after the second antibody binding step. In some embodiments, the second antibody removal step and the second enzyme substrate contact step are performed prior to the first antibody binding step. In some embodiments, the methods further comprise a second antibody stripping step comprising dissociating the second antibody from the second target epitope, wherein the second antibody stripping step is performed prior to the first antibody binding step. In some embodiments, the methods further comprise a second enzyme inactivation step comprising inactivating the second enzyme molecule, wherein the second enzyme inactivation step is performed prior to the first antibody binding step.

In some embodiments, the first enzyme molecule and the second enzyme molecule are the same. In some embodiments, the first antibody binding step is performed prior to the second antibody binding step. In some embodiments, the first antibody removal step and the first enzyme substrate contact step are performed prior to the second antibody binding step. In some embodiments, the methods further comprise a first antibody stripping step comprising dissociating the first antibody from the first target epitope, wherein the first antibody stripping step is performed prior to the second antibody binding step. In some embodiments, the methods further comprise a first enzyme inactivation step comprising inactivating the first enzyme molecule, wherein the first enzyme inactivation step is performed prior to the second antibody binding step. In some embodiments, the first antibody binding step is performed after the second antibody binding step. In some embodiments, the second antibody removal step and the second enzyme substrate contact step are performed prior to the first antibody binding step. In some embodiments, the methods further comprise a second antibody stripping step comprising dissociating the second antibody from the second target epitope, wherein the second antibody stripping step is performed prior to the first antibody binding step. In some embodiments, the methods further comprise a second enzyme inactivation step comprising inactivating the second enzyme molecule, wherein the second enzyme inactivation step is performed prior to the first antibody binding step.

In another aspect, the present invention provides methods for visualizing a cellular feature and detecting a first target epitope in a sample, the methods comprising: (a) a chemical staining step comprising contacting the sample with a chemical stain; and (b) a first antibody binding step comprising contacting the sample with a first polymeric-enzyme/antibody conjugate comprising a plurality of a first enzyme molecule and a first antibody recognizing a first target epitope in a condition suitable for forming a first complex comprising the first target epitope and the first polymeric-enzyme/antibody conjugate, thereby allowing for visualization of the cellular feature and detection of the first target epitope. In some embodiments, the chemical stain is hematoxylin and eosin (H&E) stain, papanicolaou (PAP) stain, giemsa stain, alican blue stain, mucicarmine stain, periodic acid-Schiff (PAS) stain, Masson's trichrome stain, Jone's stain, Hall's stain, iron-based stain, and Luxol fast blue stain.

In another aspect, the present invention provides methods for detecting a first target epitope in a sample, the methods comprising: a first antibody binding step comprising contacting the sample with a first polymeric-enzyme/antibody conjugate comprising a plurality of a first enzyme molecule and a first antibody recognizing a first target epitope in a condition suitable for forming a first complex comprising the first target epitope and the first polymeric-enzyme/antibody conjugate, wherein the sample is a cytology sample, thereby allowing for detection of the first target epitope. In some embodiments, the sample is a clinical smear sample, a core needle biopsy sample, a fine needle biopsy sample, or a touch imprint sample.

In some embodiments, the methods further comprises detecting a second target epitope in the sample, the method comprising: a second antibody binding step comprising contacting the sample with a second polymeric-enzyme/antibody conjugate comprising a plurality of a second enzyme molecule and a second antibody recognizing the second target epitope in a condition suitable for forming a second complex comprising the second target epitope and the second polymeric-enzyme/antibody conjugate, thereby allowing for detection of the second target epitope. In some embodiments, the methods further comprise a first enzyme substrate contact step comprising contacting the sample with a first enzyme substrate composition for the first enzyme molecule; and a second enzyme substrate contact step comprising contacting the sample with a second enzyme substrate composition for the second enzyme molecule.

In some embodiments, the first enzyme substrate composition comprises a substrate and a chromogenic substrate, a chemiluminescent substrate, a fluorogenic substrate, or a combination thereof. In some embodiments, the second enzyme substrate composition comprises a substrate and a chromogenic substrate, a chemiluminescent substrate, a fluorogenic substrate, or a combination thereof.

In some embodiments, the methods further comprise a first detection step comprising detecting a first substrate reporter generated from the first enzyme substrate composition by the first enzyme molecule.

In some embodiments, the methods further comprise a second detection step comprising detecting a second substrate reporter generated from the second enzyme substrate composition by the second enzyme molecule.

In some embodiments, the first antibody binding step or the second antibody binding step comprises incubating the first polymeric-enzyme/antibody conjugate or the second polymeric-enzyme/antibody conjugate, respectively, with the sample. In some embodiments, the first binding step or the second binding step is performed for an incubation period of about 3 minutes to about 30 minutes. In some embodiments, the first binding step or the second binding step is performed at an incubation temperature of between about 15° C. to about 37° C.

In some embodiments, the first antibody removal step or the second antibody removal step comprises contacting the sample with a wash buffer. In some embodiments, the first antibody removal step or the second antibody removal step is performed for an incubation period of about 1 minute and about 60 minutes. In some embodiments, the first antibody removal step or the second antibody removal step is performed 1-10 times. In some embodiments, the first antibody removal step or the second antibody removal step is performed at an incubation temperature of between about 15° C. and about 50° C.

In some embodiments, the first enzyme substrate composition or the second enzyme substrate composition is a solution.

In some embodiments, the first enzyme substrate contact step or the second enzyme substrate contact step is performed for an incubation period of about 1 minute and about 60 minutes. In some embodiments, the first enzyme substrate contact step or the second enzyme substrate contact step is performed at an incubation temperature of between about 15° C. and about 50° C.

In some embodiments, the methods further comprise a blocking step prior to the first antibody binding step and/or the second antibody binding step, wherein the blocking step comprises contacting the tissue with a blocking agent. In some embodiments, the blocking agent comprises skim milk, BSA, cold fish skin gelatin, casein, or an animal serum.

In some embodiments, the sample is a frozen sample. In some embodiments, the sample is fixed in a fixing solution comprising an aldehyde. In some embodiments, the fixing solution comprises formalin. In some embodiments, the sample is paraffin-embedded. In some embodiments, the sample is a formalin-fixed-paraffin-embedded sample. In some embodiments, the sample is a tissue section. In some embodiments, the tissue section is about 1.5 μm to about 5.5 μm thick. In some embodiments, the sample is a cell block section. In some embodiments, the cell block section is about 1.5 μm to about 5.5 μm thick. In some embodiments, the sample is a fresh tissue sample.

In some embodiments, the first enzyme molecule or the second enzyme molecule is selected from the group consisting of: beta-D-galactosidase, glucose oxidase, horseradish peroxidase, alkaline phosphatase, beta-lactamase, glucose-6-phosphate dehydrogenase, urease, uricase, superoxide dismutase, luciferase, pyruvate kinase, lactate dehydrogenase, galactose oxidase, acetylcholine-sterase, enterokinase, tyrosinase, and xanthine oxidase.

In some embodiments, the polymeric-enzyme/antibody conjugate comprises at least 6 enzyme molecules per polymeric-enzyme/antibody conjugate. In some embodiments, the polymeric-enzyme/antibody conjugate comprises between about 6 and about 80 enzyme molecules per polymeric-enzyme/antibody conjugate. In some embodiments, the enzyme molecules of the polymeric-enzyme are covalently linked. In some embodiments, the polymeric-enzyme has a molecular weight of about 500 kDa to about 5 MDa. In some embodiments, the polymeric-enzyme/antibody conjugate has an antibody to enzyme ratio of greater than about 1:6.

In some embodiments, the antibody is a therapeutic antibody.

In another aspect, the present invention provides kits comprising: (a) a first polymeric-enzyme/antibody conjugate comprising a plurality of a first enzyme molecule and a first antibody recognizing a first target epitope; (b) a second polymeric-enzyme/antibody conjugate comprising a plurality of a second enzyme molecule and a second antibody recognizing a second target epitope, wherein the first target epitope and the second target epitope are different.

In another aspect, the present invention provides kits comprising: (a) a first polymeric-enzyme/antibody conjugate comprising a plurality of a first enzyme molecule and a first antibody recognizing a first target epitope; (b) a chemical stain.

In some embodiments, the kits further comprise instructions for use according to the methods described herein.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIGS. 1A-1E show schematics of the direct multiplexed IHC and ICC methods described herein.

FIGS. 2A-2B show representative images of duplex direct IHC staining of frozen skin tissue.

FIG. 3 shows representative images of direct IHC staining of FFPE breast cancer tissue.

FIG. 4 shows a representative image of direct IHC staining of a tissue section that was previously stained by hematoxylin and eosin.

### DETAILED DESCRIPTION OF THE INVENTION

The present disclosure provides, in some aspects, methods, compositions, and kits for direct IHC staining applications to multiplex assays, chemically stained samples, cytology samples, and combinations thereof.

The multiplexed direct IHC methods for detecting a plurality of target epitopes in a sample eliminates the number of time-consuming steps in conventional multiplexing methods, which may allow for improvements in IHC method simplicity, time efficiency, sensitivity, and co-localization and co-referencing of a plurality of different types of markers. The direct IHC methods described herein, including multiplexed direct IHC methods, may also be applied to cytology samples and samples that have previously been chemically stained for visualization of a cellular feature. The methods disclosed herein may be used to provide point of care assessments, such as intraoperative assessments, of samples (such as surgically removed samples) useful for improving patient treatment and outcome. Moreover, the multiplexing direct IHC methods described herein may improve assessment and identification of tissue and cellular components via targeting a plurality of target epitopes and/or visualization of cellular features via chemical staining, as compared to an IHC assay performed targeting a single target epitope.

In some embodiments, the methods described herein provide an in situ diagnostic.

In some embodiments, the methods described herein may be completed within about 60 minutes or less, such as about any of 55 minutes or less, 50 minutes or less, 45 minutes or less, 40 minutes or less, 35 minutes or less, 30 minutes or less, 25 minutes or less, 20 minutes or less, or 15 minutes or less, from the time the sample was obtained from an individual, such as a human.

In some embodiments, the methods described herein may detect a target epitope, wherein the copy number of the target epitope is about 1×10⁴ or less per cell, such as 1×10³ or less per cell.

In some embodiments, the methods described herein may specifically stain frozen section or cells with a plurality of distinct colors for time-sensitive tissue or cell captures for subsequent expression analyses with minimal proteolytic-artifacts.

In some embodiments, the methods described herein may specifically stain fine needle aspiration biopsy cell smears, scrape cell smears, and touch imprinting cells, for rapid on-site assessments (e.g., at the point-of-care) with a plurality of distinct colors.

In some embodiments, the methods described herein may specifically stain enriched or extracted cells from body fluid, e.g., urine, blood, sweat, sputum, salivary, tear, and/or feces with a plurality of distinct colors.

In some embodiments, the methods described herein may specifically stain circulating tumor cells with plurality of distinct colors, where samples are of limited quantity, e.g., often limited to only one or a few slides.

In some embodiments, the methods described herein may specifically stain sub-cellular components, such as exosomes or micro-vesicles with plurality of distinct colors in time-sensitive manners.

In some embodiments, the methods described herein may specifically stain tissue sections or cytological samples that have been previously labeled with one or more specific immune florescent tags that emit different visible light spectrum upon excitations.

### Definitions

The term “antibody,” as used herein, is used in the broadest sense and encompasses various antibody structures, including but not limited to monoclonal antibodies, polyclonal antibodies, multispecific antibodies (e.g., bispecific antibodies), and antibody fragments, so long as they exhibit the desired epitope-binding activity and comprise an Fc region, a region equivalent to the Fc region of an immunoglobulin, or a region which may be useful for association of a polymeric enzyme. The terms “full-length antibody,” “intact antibody,” and “whole antibody” are used herein interchangeably to refer to an antibody having a structure substantially similar to a native antibody structure or having heavy chains that contain an Fc region as defined herein. An antibody or antibody fragment may be conjugated or otherwise derivatized within the scope of the claimed subject matter. Such antibodies include IgG1, lgG2a, IgG3, IgG4 (and IgG4 subforms), as well as IgA isotypes.

The term “native antibodies,” as used herein, is meant naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The light chain of an antibody may be assigned to one of two types, called kappa (κ) and lambda (λ), based on the amino acid sequence of its constant domain.

The term “antibody fragment,” as used herein, is meant a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g., scFv), single-domain antibodies, and multispecific antibodies formed from antibody fragments. For a review of certain antibody fragments, see, Hudson et al., Nat Med, 9, 129-134 (2003). For a review of scFv fragments, see, Pluckthun, The Pharmacology of Monoclonal Antibodies, 113, Rosenburg and Moore eds., Springer-Verlag, New York, pp. 269-315 (1994); see also WO93/16185 and U.S. Pat. Nos. 5,571,894 and 5,587,458. For discussion of Fab and F(ab′)2 fragments comprising salvage receptor binding epitope residues and having increased in vivo half-life, see, U.S. Pat. No. 5,869,046. Diabodies are antibody fragments with two antigen-binding sites that may be bivalent or bispecific. See, for example, EP404,097; WO1993/01161; Hudson et al., Nat Med, 9, pp. 129-134 (2003); and Hollinger et al., Proc Natl Acad Sci, 90, pp. 6444-6448 (1993). Triabodies and tetrabodies are also described in Hudson et al., Nat Med, 9, pp. 129-134 (2003). Single-domain antibodies are antibody fragments comprising all or a portion of the heavy chain variable domain or all or a portion of the light chain variable domain of an antibody. In some embodiments, the single-domain antibody is a human single-domain antibody (Domantis, Inc., Waltham, Mass.; see, e.g., U.S. Pat. No. 6,248,516). Antibody fragments can be made by various techniques, including but not limited to proteolytic digestion of an intact antibody as well as production by recombinant host cells (e.g., E. coli or phage).

The term “antigen binding domain,” as used herein, is meant the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable region (VL) and an antibody heavy chain variable region (VH).

The term “variable region” or “variable domain,” as used herein, is meant the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., pp. 91 (2007). A single VH or VL domain may be sufficient to confer antigen-binding specificity.

The term “hypervariable region” or “HVR,” as use herein, is meant each of the regions of an antibody variable domain which are hypervariable in sequence and/or form structurally defined loops, “hypervariable loops.” Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196, pp. 901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

In some embodiments, antibodies encompassed by the present disclosure include, for example, a chimeric antibody, a humanized antibody, a human antibody, and an antibody fusion protein.

The term “chimeric antibody,” as used herein, is meant a recombinant protein that contains the variable domains of both the heavy and light antibody chains, including the complementarity determining regions (CDRs) of an antibody derived from one species, preferably a rodent antibody, more preferably a murine antibody, while the constant domains of the antibody molecule are derived from those of a human antibody. For veterinary applications, the constant domains of the chimeric antibody may be derived from that of other species, such as a subhuman primate, cat, or dog.

The term “humanized antibody,” as used herein, is meant a recombinant protein in which the CDRs from an antibody from one species; e.g., a rodent antibody, are transferred from the heavy and light variable chains of the rodent antibody into human heavy and light variable domains. The constant domains of the antibody molecule are derived from those of a human antibody. In some embodiments, specific residues of the framework region of the humanized antibody, particularly those that are touching or close to the CDR sequences, may be modified, for example replaced with the corresponding residues from the original rodent, subhuman primate, or other antibody.

The term “human antibody,” as used herein, is meant an antibody obtained, for example, from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet, 7 (1994), Lonberg et al., Nature 368, (1994), and Taylor et al., Int Immun, 6: (1994). A fully human antibody also can be constructed by genetic or chromosomal transfection methods, as well as phage display technology, all of which are known in the art. See, for example, McCafferty et al., Nature 348, pp. 552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g., Johnson and Chiswell, Current Opinion in Structural Biology, 3, pp. 5564-571 (1993). Human antibodies may also be generated by in vitro activated B cells. See, U.S. Pat. Nos. 5,567,610 and 5,229,275, which are incorporated herein by reference in their entirety.

The term “antibody fusion protein,” as used herein, is meant a recombinantly-produced antigen-binding molecule in which two or more of the same or different natural antibody, single-chain antibody or antibody fragment segments with the same or different specificities are linked. A fusion protein comprises at least one specific binding site. Valency of the fusion protein indicates the total number of binding arms or sites the fusion protein has to antigen(s) or epitope(s); e.g., monovalent, bivalent, trivalent, or mutlivalent. The multivalency of the antibody fusion protein means that it can take advantage of multiple interactions in binding to an antigen, thus increasing the avidity of binding to the antigen, or to different antigens. Specificity indicates how many different types of antigen or epitope an antibody fusion protein is able to bind; e.g., monospecific, bispecific, trispecific, multispecific. Using these definitions, a natural antibody, e.g., an IgG, is bivalent because it has two binding arms but is monospecific because it binds to one type of antigen or epitope. A monospecific, multivalent fusion protein has more than one binding site for the same antigen or epitope. For example, a monospecific diabody is a fusion protein with two binding sites reactive with the same antigen. The fusion protein may comprise a multivalent or multispecific combination of different antibody components or multiple copies of the same antibody component. The fusion protein may additionally comprise a therapeutic agent.

As used herein, “treat,” “treatment,” or “treating” is an approach for obtaining beneficial or desired results including clinical results. For purposes of this invention, beneficial or desired clinical results include, but are not limited to, one or more of the following: alleviating one or more symptoms resulting from the disease, decreasing the dose of one or more other medications required to treat the disease, and/or increasing the quality of life.

The term “effective amount,” as used herein, refers to an amount of a compound or composition sufficient to treat a specified disorder, condition or disease such as ameliorate, palliate, lessen, and/or delay one or more of its symptoms.

As used herein, the term “individual” refers to a mammal and includes, but is not limited to, human, bovine, horse, feline, canine, rodent, or primate. In some embodiments, the individual is human.

The term “comprises” and grammatical equivalents thereof are used herein to mean that other components, ingredients, steps, etc. are optionally present. For example, an article “comprising” components A, B, and C can consist of (i.e., contain only) components A, B, and C, or can contain not only components A, B, and C but also one or more other components. It is understood that “comprises” and grammatical equivalents thereof include “consisting of” “consisting essentially of.”

Where a range of value is provided, it is understood that each intervening value, to the tenth of the unit of the lower limit unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, the singular forms “a,” “or,” and “the” include plural referents unless the context clearly dictates otherwise.

### Detecting a Plurality of Target Epitopes in a Sample

The present disclosure provides methods for detecting a plurality of target epitopes in a sample, herein referred to as multiplexed methods, such as multiplexed IHC and multiplexed ICC. Generally, the multiplexed methods described herein comprise, in sequential order for the application of each polymeric-enzyme/antibody conjugate (polyEnzyme-Ab) to a sample: (a) an antibody binding step, wherein a polymeric-enzyme/antibody conjugate is contacted with the sample; (b) an antibody removal step, wherein the polymeric-enzyme/antibody conjugates that do not form a complex with a target epitope are removed from the sample; and (c) an enzyme substrate contact step, wherein the enzyme molecules of the polymeric-enzyme are contacted with an enzyme substrate composition, e.g., a composition comprising a substrate and a luminescent substrate (such as a chemiluminescent substrate), a chromogenic substrate, a fluorogenic substrate, or a combination thereof. As described in more detail herein, the order of steps of multiplexed methods (and use of additional steps such as an antibody stripping step or an enzyme inactivation step) may depend on the polymeric-enzyme/antibody conjugates used and/or whether a first polymeric-enzyme/antibody conjugate and a second polymeric-enzyme/antibody conjugate may simultaneously or sequentially be contacted with a sample to allow for the differentiation of a plurality of target epitopes. In some embodiments, the process may be repeated multiple times to stain different epitopes with the same of different colors to achieve increased multiplexing. The paragraphs directly below describe a duplex method, however, the concepts described also apply to multiplexed methods wherein more than two polymeric-enzyme/antibody conjugates are used to probe for target epitopes in a sample.

The multiplexed methods described herein for detecting a plurality of target epitopes in a sample comprise: (a) a first antibody binding step comprising contacting the sample with a first polymeric-enzyme/antibody conjugate comprising a plurality of a first enzyme molecule and a first antibody recognizing a first target epitope in a condition suitable for forming a first complex comprising the first target epitope and the first polymeric-enzyme/antibody conjugate; (b) a second antibody binding step comprising contacting the sample with a second polymeric-enzyme/antibody conjugate comprising a plurality of a second enzyme molecule and a second antibody recognizing a second target epitope in a condition suitable for forming a second complex comprising the second target epitope and the second polymeric-enzyme/antibody conjugate; (c) a first antibody removal step comprising substantially removing from the sample the first polymeric-enzyme/antibody conjugate that does not form the first complex; (d) a second antibody removal step comprising substantially removing from the sample the second polymeric-enzyme/antibody conjugate that does not form the second complex; (e) a first enzyme substrate contact step comprising contacting the sample with a first enzyme substrate composition comprising a substrate for the first enzyme molecule; and (f) a second enzyme substrate contact step comprising contacting the sample with a second enzyme substrate composition comprising a substrate for the second enzyme molecule, thereby allowing for detection of the plurality of target epitopes in the sample via, e.g., distinct colors.

In some embodiments, the first antibody binding step and the second antibody binding step are performed simultaneously. In some embodiments, wherein two or more antibody binding steps are performed, the methods comprise performing all antibody binding steps prior to another downstream method step, e.g., an antibody removal step. For example, in some embodiments, prior to performing an antibody removal step, the methods comprise performing a first antibody binding step and a second antibody binding step.

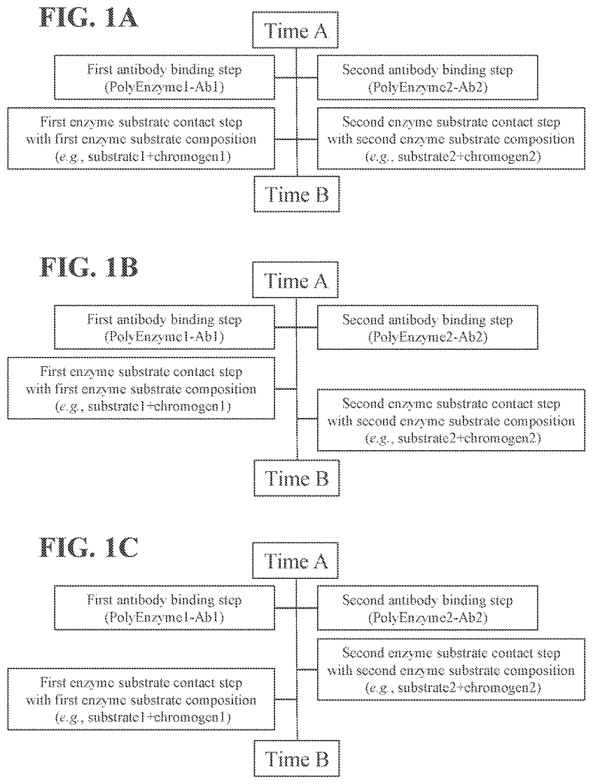

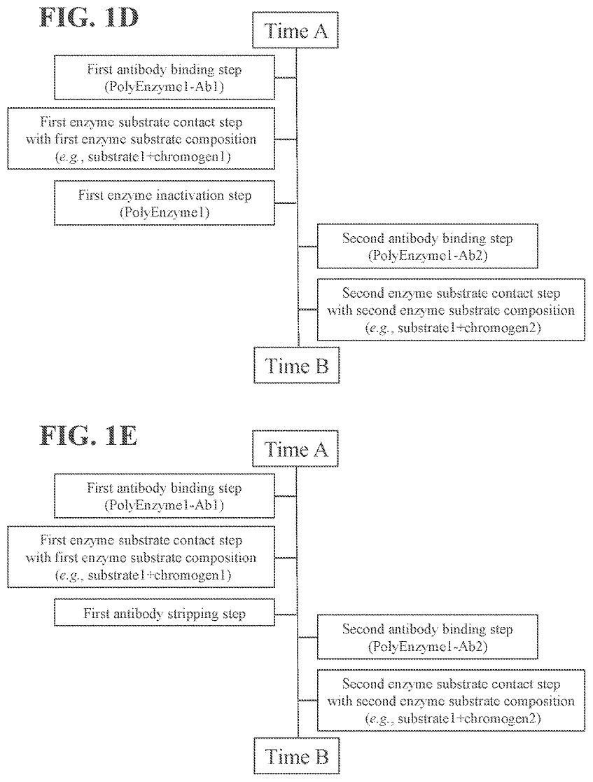

In some embodiments, wherein the first antibody binding step and the second antibody binding step are performed simultaneously, the first enzyme molecule and the second enzyme molecule are different and the first enzyme substrate composition and the second enzyme substrate composition are different. For example, as shown in FIGS. 1A-1C, the first antibody binding step and the second antibody binding step may be performed simultaneously. Subsequently, following simultaneous performance of a first antibody removal step and a second antibody removal step, a first enzyme substrate contact step and a second enzyme substrate contact step may be performed simultaneously (FIG. 1A) or sequentially (FIG. 1B and FIG. 1C). In some embodiments, simultaneously performing a first enzyme substrate contact step and a second enzyme substrate contact step, and subsequent detections step, may allow for the most time efficient assay. In some embodiments, the first enzyme substrate contact step is performed before the second enzyme substrate contact step. In some embodiments, the first enzyme substrate contact step is performed after the second enzyme substrate contact step.

Methods wherein the first enzyme molecule and the second enzyme molecule are different may also allow for sequential antibody binding steps performed, e.g., as discussed below. However, in some embodiments, methods wherein the first enzyme molecule and the second enzyme molecule are different may not require an intervening enzyme inactivation step or an antibody stripping step as discussed below.

In some embodiments, the first antibody binding step and the second antibody binding step are performed sequentially (e.g., FIG. 1D and FIG. 1E). In some embodiments, wherein the first antibody binding step and the second antibody binding step are performed sequentially, the first enzyme molecule of a first polymeric-enzyme and the second enzyme molecule of a second polymeric-enzyme are the same. In some embodiments, to allow for detection of multiple target epitopes a first enzyme inactivation step (to inactive the first enzyme molecule) is performed after a first antibody binding step and a first enzyme substrate contact step, and before a second antibody binding step (FIG. 1D). In some embodiments, to allow for detection of multiple target epitopes a first enzyme stripping step (to remove the first polymeric-enzyme antibody conjugate from the sample) is performed after a first antibody binding step and a first enzyme substrate contact step, and before a second antibody binding step (FIG. 1E).

In some embodiments, wherein the first antibody binding step and the second antibody binding step are performed sequentially, the first enzyme molecule of a first polymeric-enzyme and the second enzyme molecule of a second polymeric-enzyme are different.

The multiplexed IHC and ICC methods disclosed herein may be used to identify any number of target epitopes in a sample. In some embodiments, the number of target epitopes probed for in a method comprises at least about 2 target epitopes, such as at least about any of 3, 4, 5, 6, 7, 8, 9, or 10 target epitopes. In some embodiments, characteristics of the methods used may limit the degree of multiplexing. For example, unlike immunofluorescent-based multiplexed labeling, which can support a higher degree of multiplexing as different images capturing different wavelengths of light can be readily superimposed to create a final multiplexed image, other techniques using, e.g., chromogenic substrates, colors which are less transparent and may not support such a degree of multiplexing on a single sample. In some embodiments, wherein the sample comprises enriched and isolated cells, the constraint of overlapping colors may be reduced by increasing cell-cell separation distance.

In some embodiments, more than one of a plurality of target epitopes are of the same target antigen, e.g., the same protein. In some embodiments, each of a plurality of target epitopes is of a different target antigen, e.g., a protein.

### Visualizing a Cellular Feature and Detecting a First Target Epitope in a Sample

The present disclosure provides methods for visualizing a cellular feature and detecting a first target epitope in a sample. In some embodiments, the method comprises (a) a chemical staining step comprising contacting the sample with a chemical stain; and (b) a first antibody binding step comprising contacting the sample with a first polymeric-enzyme/antibody conjugate comprising a plurality of a first enzyme molecule and a first antibody recognizing a first target epitope in a condition suitable for forming a first complex comprising the first target epitope and the first polymeric-enzyme/antibody conjugate, thereby allowing for visualization of the cellular feature and detection of the first target epitope. In some embodiments, the chemical staining step is performed prior to a first antibody binding step. In some embodiments, the sample is pre-stained with a chemical stain. In some embodiments, the sample is stained with a chemical stain after a first antibody binding step or a second antibody binding step.

The methods for visualizing a cellular feature and detecting a first target epitope in a sample further comprise the steps disclosed herein for detecting the first target epitope. In some embodiments, the method for visualizing a cellular feature and detecting a first target epitope in a sample further comprise a chemical stain removal step.

In some embodiments, the method for visualizing a cellular feature and detecting a first target epitope in a sample further comprises a first antibody removal step comprising substantially removing from the sample the first polymeric-enzyme/antibody conjugate that does not form the first complex. In some embodiments, the method for visualizing a cellular feature and detecting a first target epitope in a sample further comprises a first enzyme substrate contact step comprising contacting the sample with a first enzyme substrate composition for the first enzyme molecule. In some embodiments, the method visualizing a cellular feature and detecting a first target epitope in a sample further comprises a first detection step comprising detecting a first substrate reporter generated from the first enzyme substrate composition by the first enzyme molecule.

In some embodiments, the methods for visualizing a cellular feature and detecting a first target epitope in a sample further comprise detecting one or more target epitopes in the sample (e.g., as described in the multiplexed methods herein). In some embodiments, the method for visualizing a cellular feature and detecting a first target epitope in a sample further comprises detecting a second target epitope in the sample, the method comprising: a second antibody binding step comprising contacting the sample with a second polymeric-enzyme/antibody conjugate comprising a plurality of a second enzyme molecule and a second antibody recognizing the second target epitope in a condition suitable for forming a second complex comprising the second target epitope and the second polymeric-enzyme/antibody conjugate, thereby allowing for detection of the second target epitope. In some embodiments, the method further comprises a second enzyme substrate contact step comprising contacting the sample with a second enzyme substrate composition for the second enzyme molecule. In some embodiments, the method further comprises a second detection step comprising detecting a second substrate reporter generated from the second enzyme substrate composition by the second enzyme molecule.

In some embodiments, the methods for visualizing a cellular feature and detecting a first target epitope in a sample further comprise a first antibody stripping step. In some embodiments, the methods for visualizing a cellular feature and detecting a first target epitope in a sample further comprise a first enzyme inactivation step.

In some embodiments, the chemical stain is one or more of hematoxylin and eosin stain (H&E), Papanicolaou stain (PAP), Giemsa, Alican Blue, Mucicarmine, PAS (glycogen, basement membrane), Masson's Trichrome (muscle, collagen), Jone's (basement membrane), Hall's (bile), Iron (hemosiderin), PAS (fungus), and Luxol Fast Blue (myelin).

Chemical staining techniques are known in the art. For example, after preparation of the sample, such as a tissue section, the sample may be placed on a slide and then stained with a chemical stain. In some embodiments, the sample is stained chemically stained with one or more chemical stains. In some embodiments, the one or more chemical stains distinctly stains different cellular components so the different cellular components may be distinguished from one another. In some embodiments, xanthine dye or the functional equivalent thereof and/or a thiazine dye or the functional equivalent thereof are used to enhance and make distinguishable the nucleus, cytoplasm, and “granular” structures within each tissue section. Such dyes are commercially available and often sold as sets. By way of example, HEMA 3® (CMS, Houston, Tex.) stain set comprises xanthine dye and thiazine dye. In some embodiments, methyleneblue may also be used. Examples of other morphological stains that may be used on the instant method include, but are not limited to, dyes that do not significantly autofluoresce at the same wavelength as another fluorescent label. One of skill in the art will appreciate that staining may be optimized for a given tissue by increasing or decreasing the length of time the slides remain in the dye.

In some embodiments, the present disclosure provides methods for determining morphology and a target epitope expression in a sample simultaneously.

In some embodiments, the sample is chemically stained and then stored for later performance of an immune-based staining method described herein. In some embodiments, the sample is an archive sample. In some embodiments, the sample is an archived sample that was previously chemically stained. In some embodiments, the sample is stored for at least about 1 month, such as about 6 months, about 1 year, about 2 years, and about 5 years, prior to performance of an immune-based staining method described herein.

In some embodiments, the incubation period of a chemical staining step is about 1 minute to about 60 minutes, such as any of: about 3 minutes to about any of 5 minutes, 10 minutes, 15 minutes, 20 minutes, 25 minutes, 30 minutes, 35 minutes, 40 minutes, 45 minutes, 50 minutes, or 55 minutes; about 5 minutes to about any of 10 minutes, 15 minutes, 20 minutes, 25 minutes, 30 minutes, 35 minutes, 40 minutes, 45 minutes, 50 minutes, or 55 minutes; about 10 minutes to about any of 15 minutes, 20 minutes, 25 minutes, 30 minutes, 35 minutes, 40 minutes, 45 minutes, 50 minutes, or 55 minutes; about 15 minutes to about any of 20 minutes, 25 minutes, 30 minutes, 35 minutes, 40 minutes, 45 minutes, 50 minutes, or 55 minutes; about 20 minutes to about any of 25 minutes, 30 minutes, 35 minutes, 40 minutes, 45 minutes, 50 minutes, or 55 minutes; about 25 minutes to about any of 30 minutes, 35 minutes, 40 minutes, 45 minutes, 50 minutes, or 55 minutes; about 30 minutes to about any of 35 minutes, 40 minutes, 45 minutes, 50 minutes, or 55 minutes; about 35 minutes to about any of 40 minutes, 45 minutes, 50 minutes, or 55 minutes; about 40 minutes to about any of 45 minutes, 50 minutes, or 55 minutes; about 45 minutes to about 50 minutes or about 55 minutes; or about 50 minutes to about 55 minutes.

In some embodiments, the chemical staining step is performed at an incubation temperature of about 10° C. to about 50° C., such as any of: about 15° C. to about any of 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 20° C. to about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 25° C. to about any of 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 4820 C., 49° C., or 50° C.; about 30° C. to about any of 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; or about 35° C. to about any of 36° C., 37° C., 38° C., 39° C., 40° C., 41° C., 42° C., 43° C., 44° C., 45° C., 46° C., 47° C., 48° C., 49° C., or 50° C.

### Detecting a First Target Epitope in a Cytology Sample

The present disclosure provides methods for detecting a first target epitope in a sample, wherein the sample is a cytology sample. In some embodiments, the method for detecting a first target epitope in a cytology sample comprises a first antibody binding step comprising contacting the sample with a first polymeric-enzyme/antibody conjugate comprising a plurality of a first enzyme molecule and a first antibody recognizing a first target epitope in a condition suitable for forming a first complex comprising the first target epitope and the first polymeric-enzyme/antibody conjugate, thereby allowing for detection of the first target epitope. In some embodiments, the first target epitope is a native epitope.

The methods for detecting a first target epitope in a cytology sample further comprise the steps disclosed herein useful for allowing detection of the first target epitope. In some embodiments, the method for detecting a first target epitope in a cytology sample further comprises a first antibody removal step comprising substantially removing from the cytology sample the first polymeric-enzyme/antibody conjugate that does not form the first complex. In some embodiments, the method for detecting a first target epitope in a cytology sample further comprises a first enzyme substrate contact step comprising contacting the sample with a first enzyme substrate composition for the first enzyme molecule. In some embodiments, the method for detecting a first target epitope in a cytology sample further comprises a first detection step comprising detecting a first substrate reporter generated from the first enzyme substrate composition by the first enzyme molecule.

In some embodiments, the methods for detecting a first target epitope in a cytology sample further comprise detecting one or more target epitopes in the cytology sample (e.g., as discussed in the multiplexed disclosure herein). In some embodiments, the method for detecting a first target epitope in a cytology sample further comprises detecting a second target epitope in the cytology sample, the method comprising: a second antibody binding step comprising contacting the sample with a second polymeric-enzyme/antibody conjugate comprising a plurality of a second enzyme molecule and a second antibody recognizing the second target epitope in a condition suitable for forming a second complex comprising the second target epitope and the second polymeric-enzyme/antibody conjugate, thereby allowing for detection of the second target epitope. In some embodiments, the method further comprises a second enzyme substrate contact step comprising contacting the sample with a second enzyme substrate composition for the second enzyme molecule. In some embodiments, the method further comprises a second detection step comprising detecting a second substrate reporter generated from the second enzyme substrate composition by the second enzyme molecule.

In some embodiments, the methods for detecting a first target epitope in a cytology sample further comprise a chemical staining step for visualization of a cellular feature.

In some embodiments, the methods for detecting a first target epitope in a cytology sample further comprise detecting one or more target epitopes in the cytology sample (e.g., as discussed in the multiplex section above) and a chemical staining step for visualization of a cellular feature.

The methods for detecting a first target epitope in a cytology sample are useful for assessing any cytology samples. In some embodiments, the cytology sample comprises a clinical smear, a fresh tissue smear, a fresh tissue sample obtained via touch imprint, fresh cells obtained from a circulating isolation process, fresh cells obtained from touch imprinting, fresh cells obtained from cell cultures, explants, fresh cells isolated from other isolation processes, fresh micro-vesicles, exosomes, or other sub cellular organelles or fragments, a body fluid, a body secretion, bronchial alveolar lavage fluid, cerebrospinal fluid, a sputum sample, a sweat sample, a urine sample, a feces sample, or a blood sample. In some embodiments, the cytology sample is a cellular component sample. In some embodiments, the cellular component sample comprises one or more of the following: micro-vesicles, exosomes, cellular debris, a membrane fragments, and a cellular organelle, or a fragment thereof. In some embodiments, the cytology sample is a circulating cell, such as a circulating cancer cell (CTC).

In some embodiments, the methods described herein may be performed on a fresh sample, e.g., may be performed immediately after removal from a patient. In some embodiments, the methods described herein may be performed at the point-of-care. In some embodiments, the methods described herein may be performed within about 60 minutes after the sample is obtained from an individual, including within about any of 55 minutes, 50 minutes, 45 minutes, 40 minutes, 35 minutes, 30 minutes, 25 minutes, 20 minutes, 15 minutes, 10 minutes, or 5 minutes. In some embodiments, the methods described herein may be performed without first fixing the sample, e.g., freezing or formalin fixing. In some embodiments, the methods described herein may be performed on a sample that has never been frozen.

In some embodiments, the cytology sample is of limited quantity. In some embodiments, the cytology sample comprises less than about 1000 cells, such as less than about any of 500 cells, 250 cells, 100 cells, 50 cells, 25 cells, 10 cells, 5 cells, 4 cells, 3 cells, or 2 cells. In some embodiments, the cytology sample comprises about any of 5 cells, 4 cells, 3 cells, 2 cells, or 1 cell. In some embodiments, the cytology sample comprises less than about 1000 cellular components, e.g., exosomes, such as less than about any of 500 cellular components, 250 cellular components, 100 cellular components, 50 cellular components, 25 cellular components, 10 cellular components, 5 cellular components, 4 cellular components, 3 cellular components, or 2 cellular components. In some embodiments, the cytology sample comprises about any of 5 cellular components, 4 cellular components, 3 cellular components, 2 cellular components, or 1 cellular component.

In some embodiments, the cytology sample is obtained via a core or fine needle biopsy technique, a magnetic bead affinity separation technique, a filtration technique, a flow cytometry technique, a touch imprint technique, an exfoliative (mechanical/spontaneous) technique, a breast smear technique, a breast secretion technique, a bronchial alveolar lavage fluid collection technique, a bronchial brushing technique, a bronchial washing technique, a cerebrospinal fluid collection technique, a gastrointestinal tract brushing technique, a pap smear, or a sediment collection technique.

In some embodiments, the cytology sample is a pap smear. In some embodiments, the methods comprise probing for (including detecting the presence and level or absence of) one or more target epitopes in cells and/or cellular components obtained from a pap smear, e.g., the target epitopes may include p16, ki67, and PCNA. In some embodiments, the methods comprise probing for (including detecting the presence and level or absence of) one or more target epitopes in cells and/or cellular components obtained from a pap smear, e.g., the target epitopes may include p16, ki67, and PCNA, wherein the method is performed at the point-of-care within about 60 minutes of obtaining the sample from the individual.

In some embodiments, the present disclosure provides methods for visualizing a cellular feature and detecting one or more target epitopes in a cytology sample according to the methods disclosed herein. In some embodiments, the cytology sample was previously chemically stained. In some embodiments, the cytology sample is an archived sample. In some embodiments, the cytology sample is an archived sample that was previously chemically stained. In some embodiments, the cytology sample is a fresh sample. In some embodiments, the methods comprise (a) a chemical staining step comprising contacting a cytology sample with a chemical stain; (b) a first antibody binding step comprising contacting the cytology sample with a first polymeric-enzyme/antibody conjugate comprising a plurality of a first enzyme molecule and a first antibody recognizing a first target epitope in a condition suitable for forming a first complex comprising the first target epitope and the first polymeric-enzyme/antibody conjugate; (c) a second antibody binding step comprising contacting the cytology sample with a second polymeric-enzyme/antibody conjugate comprising a plurality of a second enzyme molecule and a second antibody recognizing a second target epitope in a condition suitable for forming a second complex comprising the second target epitope and the second polymeric-enzyme/antibody conjugate; (d) a first antibody removal step comprising substantially removing from the cytology sample the first polymeric-enzyme/antibody conjugate that does not form the first complex; (e) a second antibody removal step comprising substantially removing from the cytology sample the second polymeric-enzyme/antibody conjugate that does not form the second complex; (f) a first enzyme substrate contact step comprising contacting the cytology sample with a first enzyme substrate composition comprising a substrate for the first enzyme molecule; and (g) a second enzyme substrate contact step comprising contacting the cytology sample with a second enzyme substrate composition comprising a substrate for the second enzyme molecule, thereby allowing for detection of the plurality of target epitopes in the cytology sample via, e.g., distinct colors.

### Antibody Binding Step

The methods for detecting a target epitope in a sample disclosed herein, in some aspects, comprise one or more antibody binding steps.

In some embodiments, the antibody binding step comprises incubating a polymeric-enzyme/antibody conjugate, such as a first polymeric-enzyme/antibody conjugate or a second polymeric-enzyme/antibody conjugate, with a sample. In some embodiments, the polymeric-enzyme/antibody conjugate is admixed in a buffer. In some embodiments, the buffer comprises one or more of phosphate, tris, MOPS, MES, HEPES, or bicarbonate. In some embodiments, the buffer comprises phosphate-buffered saline (PBS). In some embodiments, the buffer comprises tris-buffered saline (PBS). In some embodiments, the buffer further comprises one or more of thiomersal, proclin 300, manganese, calcium, iron, magnesium, zinc, polyethylene glycol (PEG) with molecular weight from 400 to 40,000 Da, ethylene glycol, glycerol, bovine serum albumin, horse serum albumin, goat serum albumin, rabbit serum albumin, trehalose, sucrose, gelatin, Tween 20, Tween 30, dextransulfate with molecular weight from 300 to 30,000 Da, or DEAE dextran with molecular weight from 500 to 25,000 Da. In some embodiments, the buffer further comprises PEG with a molecular weight of 400, 1500, or 6000. The buffers useful in the methods disclosed herein may be optimized, e.g., by adjusting the concentration of a polymeric-enzyme/antibody conjugate or a buffer component, to increase antibody binding to a target epitope. In some embodiments, the buffer is a commercialized buffer from Novodiax, Inc. (Hayward, Calif.), e.g., product catalog #C30001.

In some embodiments, the incubation period of an antibody binding step, such as a first antibody binding step or a second antibody binding step, is about 1 minute to about 60 minutes, such as any of: about 3 minutes to about any of 5 minutes, 10 minutes, 15 minutes, 20 minutes, 25 minutes, 30 minutes, 35 minutes, 40 minutes, 45 minutes, 50 minutes, or 55 minutes; about 5 minutes to about any of 10 minutes, 15 minutes, 20 minutes, 25 minutes, 30 minutes, 35 minutes, 40 minutes, 45 minutes, 50 minutes, or 55 minutes; about 10 minutes to about any of 15 minutes, 20 minutes, 25 minutes, 30 minutes, 35 minutes, 40 minutes, 45 minutes, 50 minutes, or 55 minutes; about 15 minutes to about any of 20 minutes, 25 minutes, 30 minutes, 35 minutes, 40 minutes, 45 minutes, 50 minutes, or 55 minutes; about 20 minutes to about any of 25 minutes, 30 minutes, 35 minutes, 40 minutes, 45 minutes, 50 minutes, or 55 minutes; about 25 minutes to about any of 30 minutes, 35 minutes, 40 minutes, 45 minutes, 50 minutes, or 55 minutes; about 30 minutes to about any of 35 minutes, 40 minutes, 45 minutes, 50 minutes, or 55 minutes; about 35 minutes to about any of 40 minutes, 45 minutes, 50 minutes, or 55 minutes; about 40 minutes to about any of 45 minutes, 50 minutes, or 55 minutes; about 45 minutes to about 50 minutes or about 55 minutes; or about 50 minutes to about 55 minutes.

In some embodiments, the incubation period of an antibody binding step, such as a first antibody binding step or a second antibody binding step, is about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of an antibody binding step, such as a first antibody binding step or a second antibody binding step, is less than about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of an antibody binding step, such as a first antibody binding step or a second antibody binding step, is greater than about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of an antibody binding step, such as a first antibody binding step or a second antibody binding step, is performed at an incubation temperature of about 10° C. to about 50° C., such as any of: about 15° C. to about any of 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 20° C. to about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 25° C. to about any of 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 30° C. to about any of 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; or about 35° C. to about any of 36° C., 37° C., 38° C., 39° C., 40° C., 41° C., 42° C., 43° C., 44° C., 45° C., 46° C., 47° C., 48° C., 49° C., or 50° C.

In some embodiments, the incubation period of an antibody binding step, such as a first antibody binding step or a second antibody binding step, is performed at an incubation temperature of about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the incubation period of an antibody binding step, such as a first antibody binding step or a second antibody binding step, is performed at an incubation temperature of at least about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the incubation period of an antibody binding step, such as a first antibody binding step or a second antibody binding step, is performed at an incubation temperature of greater than about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the incubation period of an antibody binding step, such as a first antibody binding step or a second antibody binding step, is performed at one or more the incubation temperatures disclosed herein.

The first antibody binding step and the second antibody binding step may comprise any combination of conditions, e.g., incubation period and temperature. In some embodiments, wherein the first antibody binding step and the second antibody binding step are not performed simultaneously, the conditions of the first antibody binding step and the second antibody binding step are the same. In some embodiments, wherein the first antibody binding step and the second antibody binding step are not performed simultaneously, the conditions of the first antibody binding step and the second antibody binding step are different.

### Antibody Removal Step

The methods for detecting a target epitope in a sample disclosed herein, in some aspects, comprise one or more antibody removal steps. The antibody removal step may remove unbound and non-specifically bound polymeric-enzyme/antibody conjugates from the sample to reduce non-specific signals.

In some embodiments, the antibody removal step comprises contacting a sample with a wash buffer for an incubation period. In some embodiments, the wash buffer comprises one or more of PBS, TBS, MOPS, MES, HEPES, or bicarbonate buffer. In some embodiments, the wash buffer further comprises a detergent, e.g., Tween (e.g., 0.01-0.2%). In some embodiments, the wash buffer comprises 10 mM PBS with 0.05% Tween 20.

In some embodiments, the incubation period of an antibody removal step, such as a first antibody removal step or a second antibody removal step, is about 1 minute to about 60 minutes, such as any of: about 3 minutes to about any of 5 minutes, 10 minutes, 15 minutes, 20 minutes, 25 minutes, 30 minutes, 35 minutes, 40 minutes, 45 minutes, 50 minutes, or 55 minutes; about 5 minutes to about any of 10 minutes, 15 minutes, 20 minutes, 25 minutes, 30 minutes, 35 minutes, 40 minutes, 45 minutes, 50 minutes, or 55 minutes; about 10 minutes to about any of 15 minutes, 20 minutes, 25 minutes, 30 minutes, 35 minutes, 40 minutes, 45 minutes, 50 minutes, or 55 minutes; about 15 minutes to about any of 20 minutes, 25 minutes, 30 minutes, 35 minutes, 40 minutes, 45 minutes, 50 minutes, or 55 minutes; about 20 minutes to about any of 25 minutes, 30 minutes, 35 minutes, 40 minutes, 45 minutes, 50 minutes, or 55 minutes; about 25 minutes to about any of 30 minutes, 35 minutes, 40 minutes, 45 minutes, 50 minutes, or 55 minutes; about 30 minutes to about any of 35 minutes, 40 minutes, 45 minutes, 50 minutes, or 55 minutes; about 35 minutes to about any of 40 minutes, 45 minutes, 50 minutes, or 55 minutes; about 40 minutes to about any of 45 minutes, 50 minutes, or 55 minutes; about 45 minutes to about 50 minutes or about 55 minutes; or about 50 minutes to about 55 minutes.

In some embodiments, the incubation period of an antibody removal step, such as a first antibody removal step or a second antibody removal step, is about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of an antibody removal step, such as a first antibody removal step or a second antibody removal step, is less than about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of an antibody removal step, such as a first antibody removal step or a second antibody removal step, is greater than about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of an antibody removal step, such as a first antibody removal step or a second antibody removal step, is performed at an incubation temperature of about 10° C. to about 50° C., such as any of: about 15° C. to about any of 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 20° C. to about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 25° C. to about any of 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 30° C. to about any of 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; or about 35° C. to about any of 36° C., 37° C., 38° C., 39° C., 40° C., 41° C., 42° C., 43° C., 44° C., 45° C., 46° C., 47° C., 48° C., 49° C., or 50° C.

In some embodiments, the incubation period of an antibody removal step, such as a first antibody removal step or a second antibody removal step, is performed at an incubation temperature of about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the incubation period of an antibody removal step, such as a first antibody removal step or a second antibody removal step, is performed at an incubation temperature of at least about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the incubation period of an antibody removal step, such as a first antibody removal step or a second antibody removal step, is performed at an incubation temperature of greater than about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the antibody removal step, such as a first antibody removal step or a second antibody removal step, is repeated 1 to 10 times, wherein the wash buffered is removed from the sample, such as decanted, after an incubation period. In some embodiments, the antibody removal step is repeated 1 time, 2, times, 3 times, 4 times, 5 times, 6 times, 7 times, 8 times, 9 times, or 10 times.

The first antibody removal step and the second antibody removal step may comprise any combination of conditions, e.g., incubation period and temperature. In some embodiments, wherein the first antibody removal step and the second antibody removal step are not performed simultaneously, the conditions of the first antibody removal step and the second antibody removal step are the same. In some embodiments, wherein the first antibody removal step and the second antibody removal step are not performed simultaneously, the conditions of the first antibody removal step and the second antibody removal step are different. In some embodiments, wherein the first antibody removal step is repeated, the conditions of the replicates of the first antibody removal step are the same. In some embodiments, wherein the second antibody removal step is repeated, the conditions of the replicates of the second antibody removal step are different.

In some embodiments, the number of times an antibody removal step is repeated for a method disclosed herein comprising use of a polymeric-enzyme/antibody conjugate is reduced as compared to a method using an antibody conjugated with a single enzyme molecule or a primary antibody require use of a secondary antibody for detection. In some embodiments, the incubation period of an antibody removal step for a method disclosed herein comprising use of a polymeric-enzyme/antibody conjugate is reduced as compared to a method using an antibody conjugated with a single enzyme molecule or a primary antibody require use of a secondary antibody for detection. In some embodiments, the number of times an antibody removal step is repeated and the incubation period of an antibody removal step for a method disclosed herein comprising use of a polymeric-enzyme/antibody conjugate are reduced as compared to a method using an antibody conjugated with a single enzyme molecule or a primary antibody require use of a secondary antibody for detection. In some embodiments, the stringency of the washing buffer for a method disclosed herein comprising use of a polymeric-enzyme/antibody conjugate is reduced as compared to a method using an antibody conjugated with a single enzyme molecule or a primary antibody require use of a secondary antibody for detection.

### Enzyme Substrate Contact Step

The methods for detecting a target epitope in a sample disclosed herein, in some aspects, comprise one or more enzyme substrate contact steps. In some embodiments, each substrate contact step comprises contacting the sample with an enzyme substrate composition comprising a substrate that can be catalyzed by an active enzyme applied to the sample in a previous step and a luminescent substrate (such as a chemiluminescent substrate), a chromogenic substrate, a fluorogenic substrate, or a combination thereof that, after reacting with the enzyme, displays a distinct color.

In some embodiments, the enzyme substrate contact step comprises contacting a sample with one or more enzyme substrate compositions, such as a first enzyme substrate composition for a first enzyme molecule or a second enzyme substrate composition for a second enzyme molecule. In some embodiments, the enzyme substrate composition comprising a substrate and a luminescent substrate (such as a chemiluminescent substrate), a chromogenic substrate, a fluorogenic substrate, or a combination thereof. In some embodiments, the enzyme substrate composition comprises a buffer comprises one or more of phosphate, tris, MOPS, MES, HEPES, or bicarbonate. In some embodiments, the buffer comprises phosphate-buffered saline (PBS). In some embodiments, the buffer comprises tris-buffered saline (PBS). In some embodiments, the buffer further comprises one or more of thiomersal, proclin 300, manganese, calcium, iron, magnesium, zinc, polyethylene glycol (PEG) with molecular weight from 400 to 40,000 Da, ethylene glycol, glycerol, bovine serum albumin, horse serum albumin, goat serum albumin, rabbit serum albumin, trehalose, sucrose, gelatin, Tween 20, Tween 30, dextransulfate with molecular weight from 300 to 30,000 Da, or DEAE dextran with molecular weight from 500 to 25,000 Da. In some embodiments, the buffer further comprises bovine serum albumin and/or polyethylene glycol.

In some embodiments, the incubation period of an enzyme substrate contact step, such as a first enzyme substrate contact step or a second enzyme substrate contact step, is about 1 minute to about 60 minutes, such as any of: about 3 minutes to about any of 5 minutes, 10 minutes, 15 minutes, 20 minutes, 25 minutes, 30 minutes, 35 minutes, 40 minutes, 45 minutes, 50 minutes, or 55 minutes; about 5 minutes to about any of 10 minutes, 15 minutes, 20 minutes, 25 minutes, 30 minutes, 35 minutes, 40 minutes, 45 minutes, 50 minutes, or 55 minutes; about 10 minutes to about any of 15 minutes, 20 minutes, 25 minutes, 30 minutes, 35 minutes, 40 minutes, 45 minutes, 50 minutes, or 55 minutes; about 15 minutes to about any of 20 minutes, 25 minutes, 30 minutes, 35 minutes, 40 minutes, 45 minutes, 50 minutes, or 55 minutes; about 20 minutes to about any of 25 minutes, 30 minutes, 35 minutes, 40 minutes, 45 minutes, 50 minutes, or 55 minutes; about 25 minutes to about any of 30 minutes, 35 minutes, 40 minutes, 45 minutes, 50 minutes, or 55 minutes; about 30 minutes to about any of 35 minutes, 40 minutes, 45 minutes, 50 minutes, or 55 minutes; about 35 minutes to about any of 40 minutes, 45 minutes, 50 minutes, or 55 minutes; about 40 minutes to about any of 45 minutes, 50 minutes, or 55 minutes; about 45 minutes to about 50 minutes or about 55 minutes; or about 50 minutes to about 55 minutes.

In some embodiments, the incubation period of an enzyme substrate contact step, such as a first enzyme substrate contact step or a second enzyme substrate contact step, is about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of an enzyme substrate contact step, such as a first enzyme substrate contact step or a second enzyme substrate contact step, is less than about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of an enzyme substrate contact step, such as a first enzyme substrate contact step or a second enzyme substrate contact step, is greater than about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of an enzyme substrate contact step, such as a first enzyme substrate contact step or a second enzyme substrate contact step, is performed at an incubation temperature of about 10° C. to about 50° C., such as any of: about 15° C. to about any of 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 20° C. to about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 25° C. to about any of 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 30° C. to about any of 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; or about 35° C. to about any of 36° C., 37° C., 38° C., 39° C., 40° C., 41° C., 42° C., 43° C., 44° C., 45° C., 46° C., 47° C., 48° C., 49° C., or 50° C.

In some embodiments, the incubation period of an enzyme substrate contact step, such as a first enzyme substrate contact step or a second enzyme substrate contact step, is performed at an incubation temperature of about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the incubation period of an enzyme substrate contact step, such as a first enzyme substrate contact step or a second enzyme substrate contact step, is performed at an incubation temperature of at least about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the incubation period of an enzyme substrate contact step, such as a first enzyme substrate contact step or a second enzyme substrate contact step, is performed at an incubation temperature of greater than about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

The first enzyme substrate contact step and the second enzyme substrate contact step may comprise any combination of conditions, e.g., incubation period and temperature. In some embodiments, wherein the first enzyme substrate contact step and the second enzyme substrate contact step are not performed simultaneously, the conditions of the first enzyme substrate contact and the second enzyme substrate contact step are the same. In some embodiments, wherein the first enzyme substrate contact step and the second enzyme substrate contact step are not performed simultaneously, the conditions of the first enzyme substrate contact step and the second enzyme substrate contact step are different.

In some embodiments, after the incubation period of an enzyme substrate contact step, a sample is washed one or more times with water, such as tap water, prior to a detection step.

### Detection Step

The methods for detecting a target epitope in a sample disclosed herein, in some aspects, comprise one or more detection steps. In some embodiments, the detection step, such as a first detection step or a second detection step, comprises detecting a substrate reporter generated from an enzyme substrate composition. In some embodiments, the method comprises detecting a first substrate reporter generated from a first enzyme substrate composition by a first enzyme molecule. In some embodiments, the method comprises detecting a second substrate reporter generated from a second enzyme substrate composition by a second enzyme molecule.

The methods of the detection step may generally be based on the enzyme, the enzyme substrate composition, and/or substrate reporter and the type of light being detected therefrom. In some embodiments, the substrate reporter absorbs light of a defined range of wavelengths. In some embodiments, the substrate reporter emits light at a defined range of wavelengths, e.g., fluorescence emission. In some embodiments, detecting the target epitope occurs via detection of light following light excitation of the sample. In some embodiments, detecting the target epitope occurs via detection of light following a chemiluminescent reaction.

In some embodiments, the detection step, such as a first detection step or a second detection step, is performed at a temperature of about 10° C. to about 50° C., such as any of: about 15° C. to about any of 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 20° C. to about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 25° C. to about any of 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 30° C. to about any of 31° C., 32° C., 33° C., 34 ° C., 35° C., 36° C., 37° C., 38° C., 39° C., 40° C., 41° C., 42° C., 43° C., 44° C., 45° C., 46° C., 47° C., 48° C., 49° C., or 50° C.; or about 35° C. to about any of 36° C., 37° C., 38° C., 39° C., 40° C., 41° C., 42° C., 43° C., 44° C., 45° C., 46° C., 47° C., 48° C., 49° C., or 50° C.

In some embodiments, the detection step, such as a first detection step or a second detection step, is performed at a temperature of about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the detection step, such as a first detection step or a second detection step, is performed at a temperature of at least about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the detection step, such as a first detection step or a second detection step, is performed at a temperature of greater than about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

For methods comprising more than one detection step, a first detection step and a second detection step may comprise any combination of conditions, e.g., temperature. In some embodiments, wherein the first detection step and the second detection step are not performed simultaneously, the conditions of the first detection and the second detection step are the same. In some embodiments, wherein the first detection step and the second detection step are not performed simultaneously, the conditions of the first detection and the second detection step are different.

In some embodiments, the substrate reporter is detected using a spectrophotometer. In some embodiments, the substrate reporter is detected using a chemiluminometer. In some embodiments, the substrate reporter is detected using a fluorescent detector. In some embodiments, the substrate reporter is detected using a colormetric signal detector. In some embodiments, the substrate reporter is detected using a light microscope or a fluorescent microscope.

In some embodiments, the methods further comprise a quantification step. In some embodiments, the target epitope is quantified. In some embodiments, the target antigen comprising the target epitope is quantified. In some embodiments, the quantification is relative quantification. In some embodiments, the quantification is relative to a standard, such as a control sample or another portion of the sample. In some embodiments, the quantification is relative to a standard curve.

In some embodiments, wherein the method comprises a first detection step and a second detection step

### Enzyme Inactivation Step

In some embodiments, the methods for sequentially detecting a plurality of target epitopes in a sample comprise one or more enzyme inactivation steps for inactivating an enzyme molecule of a polymeric-enzyme/antibody conjugate associated with the sample in a prior step to ensure that prior applied polymeric-enzyme/antibody conjugates do not participate in subsequent catalytic reactions.

In some embodiments, the methods disclosed herein for distinguishing two or more target epitopes in a sample may require that a first enzyme molecule of a first polymeric-enzyme/antibody conjugate comprising a plurality of the first enzyme molecule and a first antibody recognizing a first target epitope that has formed a first complex be inactivated prior to a second antibody binding step or a second enzyme substrate contact step. In some embodiments, the enzyme molecule inactivation step is performed prior to a second antibody binding step. In some embodiments, the enzyme molecule inactivation step is performed prior to a second enzyme substrate contact step. In some embodiments, the first enzyme molecule and the second enzyme molecule are the same. In some embodiments, the first enzyme molecule and the second enzyme molecule are different.

In some embodiments, the enzyme molecule inactivation step is performed after a first detection step. In some embodiments, the antibody stripping step is performed after a first detection step and prior to a second antibody contact step.

For example, for the enzyme horseradish peroxidase (HRP), after color development by a first enzyme substrate composition, the sample may be treated with 5 to 60% hydrogen peroxide (H₂O₂) for 5-60 minutes and then washed with washing buffer solution to prepare the sample for detection of a target epitope with a second polymeric-enzyme/antibody conjugate. For the enzyme alkaline phosphatase, after color development by a first enzyme substrate composition, the sample may be treated with carbon dioxide mau to inactivate alkaline phosphatase. For the enzyme alkaline phosphatase, the sample may also be inactivated by heat, e.g., heating for about 10 minutes at above 65° C. or at 74° C. for about 15 minutes.

In some embodiments, the incubation period of an enzyme molecule inactivation step is about 1 minute to about 60 minutes, such as any of: about 3 minutes to about any of 5 minutes, 10 minutes, 15 minutes, 20 minutes, 25 minutes, 30 minutes, 35 minutes, 40 minutes, 45 minutes, 50 minutes, or 55 minutes; about 5 minutes to about any of 10 minutes, 15 minutes, 20 minutes, 25 minutes, 30 minutes, 35 minutes, 40 minutes, 45 minutes, 50 minutes, or 55 minutes; about 10 minutes to about any of 15 minutes, 20 minutes, 25 minutes, 30 minutes, 35 minutes, 40 minutes, 45 minutes, 50 minutes, or 55 minutes; about 15 minutes to about any of 20 minutes, 25 minutes, 30 minutes, 35 minutes, 40 minutes, 45 minutes, 50 minutes, or 55 minutes; about 20 minutes to about any of 25 minutes, 30 minutes, 35 minutes, 40 minutes, 45 minutes, 50 minutes, or 55 minutes; about 25 minutes to about any of 30 minutes, 35 minutes, 40 minutes, 45 minutes, 50 minutes, or 55 minutes; about 30 minutes to about any of 35 minutes, 40 minutes, 45 minutes, 50 minutes, or 55 minutes; about 35 minutes to about any of 40 minutes, 45 minutes, 50 minutes, or 55 minutes; about 40 minutes to about any of 45 minutes, 50 minutes, or 55 minutes; about 45 minutes to about 50 minutes or about 55 minutes; or about 50 minutes to about 55 minutes.

In some embodiments, the enzyme molecule inactivation step is performed at a temperature of about 10° C. to about 80° C., such as about 15° C. to about any of 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 78° C., 79° C., or 80° C.

### Antibody Stripping Step

In some embodiments, the methods for sequentially detecting a plurality of target epitopes in a sample comprise one or more antibody stripping steps for removing or eluting a polymeric-enzyme/antibody conjugate associated with the sample in a prior step, such as via a complex with the target epitope, to ensure that prior applied polymeric-enzyme/antibody conjugates do not participate in subsequent catalytic reactions. Generally, the antibody stripping step comprises incubating the sample under harsher conditions than regular physiological wash buffers to allow for dissociation of the complexed polymeric-enzyme/antibody conjugate.

In some embodiments, techniques for stripping an antibody from a sample use low pH (e.g., 50 mM glycine-HCl, pH 2.2), high pH (e.g., 100 mM glycine, NaOH, pH 10), osmolarity (e.g., 3.5 M KCl), detergents, denaturing agents (e.g., 25 mM glycine-HCl, 10% SDS, pH 2; or 62 mM Tris, 2% SDS, pH 6.75; or 62 mM Tris, 2% SDS, 100 mM b-mercaptoethanol, pH 6.75), and/or heat to interfere with the non-covalent binding of the polymeric-enzyme/antibody conjugate. In some embodiments, the stripping buffer comprises 50 mM glycine-HCl, pH 2.2. In some embodiments, the stripping buffer preserves the natural form of the epitope.

It is also known that some detection substrates used in IHC and ICC, such as DAB and tyramide, precipitate thus forming stronger covalent bonds on the sample. Theoretically, such agents should not be eluted by such a stripping buffer. (See, Journal of Histochemistry & Cytochemistry, Volume 57(6): 567-575, 2009).

The methods for detecting a target epitope in a sample disclosed herein, in some aspects, comprise one or more antibody stripping steps and/or enzyme inactivation steps. In some embodiments, the methods disclosed herein for distinguishing two or more target epitopes in a sample may require that a first polymeric-enzyme/antibody conjugate comprising a plurality of a first enzyme molecule and a first antibody recognizing a first target epitope that has formed a first complex be removed from a sample prior to a second antibody binding step comprising contacting the sample with a second polymeric-enzyme/antibody conjugate comprising a plurality of a second enzyme molecule and a second antibody recognizing a second target epitope. In some embodiments, the first enzyme molecule and the second enzyme molecule are the same. In some embodiments, the first enzyme molecule and the second enzyme molecule are different.

In some embodiments, the antibody stripping step is performed after a first detection step. In some embodiments, the antibody stripping step is performed after a first detection step and prior to a second antibody contact step.

In some embodiments, the incubation period of an antibody stripping step is about 1 minute to about 60 minutes, such as any of: about 3 minutes to about any of 5 minutes, 10 minutes, 15 minutes, 20 minutes, 25 minutes, 30 minutes, 35 minutes, 40 minutes, 45 minutes, 50 minutes, or 55 minutes; about 5 minutes to about any of 10 minutes, 15 minutes, 20 minutes, 25 minutes, 30 minutes, 35 minutes, 40 minutes, 45 minutes, 50 minutes, or 55 minutes; about 10 minutes to about any of 15 minutes, 20 minutes, 25 minutes, 30 minutes, 35 minutes, 40 minutes, 45 minutes, 50 minutes, or 55 minutes; about 15 minutes to about any of 20 minutes, 25 minutes, 30 minutes, 35 minutes, 40 minutes, 45 minutes, 50 minutes, or 55 minutes; about 20 minutes to about any of 25 minutes, 30 minutes, 35 minutes, 40 minutes, 45 minutes, 50 minutes, or 55 minutes; about 25 minutes to about any of 30 minutes, 35 minutes, 40 minutes, 45 minutes, 50 minutes, or 55 minutes; about 30 minutes to about any of 35 minutes, 40 minutes, 45 minutes, 50 minutes, or 55 minutes; about 35 minutes to about any of 40 minutes, 45 minutes, 50 minutes, or 55 minutes; about 40 minutes to about any of 45 minutes, 50 minutes, or 55 minutes; about 45 minutes to about 50 minutes or about 55 minutes; or about 50 minutes to about 55 minutes.

In some embodiments, the incubation period of an antibody stripping step is about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of an antibody stripping step is less than about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of an antibody stripping step is greater than about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the antibody stripping step is performed at a temperature of about 10° C. to about 50° C., such as any of: about 15° C. to about any of 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 20° C. to about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 25° C. to about any of 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 30° C. to about any of 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; or about 35° C. to about any of 36° C., 37° C., 38° C., 39° C., 40° C., 41° C., 42° C., 43° C., 44° C., 45° C., 46° C., 47° C., 48° C., 49° C., or 50° C.

In some embodiments, the antibody stripping step is performed at a temperature of about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the antibody stripping step is performed at a temperature of at least about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the antibody stripping step is performed at a temperature of greater than about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

### Blocking Step

In some embodiments, the methods of the present disclosure further comprise a blocking step prior to the first antibody binding step and/or the second antibody binding step, wherein the blocking step comprises contacting the tissue with a blocking agent. In some embodiments, the blocking step is prior to the first antibody binding step. In some embodiments, the blocking step is prior to the second antibody binding step. In some embodiments, the blocking step is prior to the first antibody binding step and the second antibody binding step. In some embodiments, the method comprises two or more blocking steps. In some embodiments, the first blocking step is prior to the first antibody binding step and a second blocking step is prior to the second antibody binding step. In some embodiments, the first blocking step and the second blocking step are the same. In some embodiments, the first blocking step and the second blocking step are different.

In some embodiments, the blocking agent comprises skim milk, BSA, cold fish skin gelatin, casein, or an animal serum. In some embodiments, the blocking agent comprises a buffer, such as TBS or PBS with BSA.

In some embodiments, the blocking agent comprises a buffer system selected from PBS, TBS, MOPS, MES, HEPES, and bicarbonate, optionally with 0.5-6% of bovine serum albumin, horse serum albumin, goat serum albumin, rabbit serum albumin, or gelatin, and 0.001-0.05% of Tween 20.

In some embodiments, the blocking agent comprises about any of 1%, 2%, 3%, 4%, 5%, 6%, 7%, 8%, 9%, or 10% skim milk.

In some embodiments, the blocking agent comprises about any of 1%, 2%, 3%, 4%, 5%, 6%, 7%, 8%, 9%, 10%, 11%, 12%, 13%, 14%, or 15% BSA.

In some embodiments, the incubation period of a blocking step is about 1 minute to about 60 minutes, such as any of: about 3 minutes to about any of 5 minutes, 10 minutes, 15 minutes, 20 minutes, 25 minutes, 30 minutes, 35 minutes, 40 minutes, 45 minutes, 50 minutes, or 55 minutes; about 5 minutes to about any of 10 minutes, 15 minutes, 20 minutes, 25 minutes, 30 minutes, 35 minutes, 40 minutes, 45 minutes, 50 minutes, or 55 minutes; about 10 minutes to about any of 15 minutes, 20 minutes, 25 minutes, 30 minutes, 35 minutes, 40 minutes, 45 minutes, 50 minutes, or 55 minutes; about 15 minutes to about any of 20 minutes, 25 minutes, 30 minutes, 35 minutes, 40 minutes, 45 minutes, 50 minutes, or 55 minutes; about 20 minutes to about any of 25 minutes, 30 minutes, 35 minutes, 40 minutes, 45 minutes, 50 minutes, or 55 minutes; about 25 minutes to about any of 30 minutes, 35 minutes, 40 minutes, 45 minutes, 50 minutes, or 55 minutes; about 30 minutes to about any of 35 minutes, 40 minutes, 45 minutes, 50 minutes, or 55 minutes; about 35 minutes to about any of 40 minutes, 45 minutes, 50 minutes, or 55 minutes; about 40 minutes to about any of 45 minutes, 50 minutes, or 55 minutes; about 45 minutes to about 50 minutes or about 55 minutes; or about 50 minutes to about 55 minutes.

In some embodiments, the incubation period of a blocking step is about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of a blocking step is less than about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the incubation period of a blocking step is greater than about any of 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 55 minutes, or 60 minutes.

In some embodiments, the blocking step is performed at a temperature of about 10° C. to about 50° C., such as any of: about 15° C. to about any of 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 20° C. to about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 25° C. to about any of 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; about 30° C. to about any of 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.; or about 35° C. to about any of 36° C., 37° C., 38° C., 39° C., 40° C., 41° C., 42° C., 43° C., 44° C., 45° C., 46° C., 47° C., 48° C., 49° C., or 50° C.

In some embodiments, the blocking step is performed at a temperature of about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the blocking step is performed at a temperature of at least about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the blocking step is performed at a temperature of greater than about any of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., or 50° C.

In some embodiments, the blocking step is performed 1, 2, 3, 4, or 5 times. In some embodiments, the blocking step is performed 1, 2, or 3 times, wherein the blocking agent comprises about 1%, 2%, 3%, 4%, 5%, 6%, or 7% skim milk, and wherein the blocking period is less than about any of 3, 4, 5, 6, 7, 8, 9, 10, 11, 12, 13, 14, 15, 16, 17, 18, 19, 20, 25, 30, 35, 40, 45, 50, 55, or 60 minutes.

In some embodiments, the blocking step is performed 1, 2, or 3 times, wherein the blocking agent comprises about 1%, 2%, 3%, 4%, 5%, 6%, or 7% skim milk, and wherein the blocking period is greater than about any of 3, 4, 5, 6, 7, 8, 9, 10, 11, 12, 13, 14, 15, 16, 17, 18, 19, 20, 25, 30, 35, 40, 45, 50, 55, or 60 minutes.

In some embodiments, the blocking step is performed 1, 2, or 3 times, wherein the blocking agent comprises about any of 1%, 2%, 3%, 4%, 5%, 6%, or 7% BSA, and wherein the blocking period is less than about any of 3, 4, 5, 6, 7, 8, 9, 10, 11, 12, 13, 14, 15, 16, 17, 18, 19, 20, 25, 30, 35, 40, 45, 50, 55, or 60 minutes.

In some embodiments, the blocking step is performed 1, 2, or 3 times, wherein the blocking agent comprises about any of 1%, 2%, 3%, 4%, 5%, 6%, or 7% BSA, and wherein the blocking period is greater than about any of 3, 4, 5, 6, 7, 8, 9, 10, 11, 12, 13, 14, 15, 16, 17, 18, 19, 20, 25, 30, 35, 40, 45, 50, 55, or 60 minutes.

In some embodiments, after the blocking step, a sample is washed one or more times with water, such as tap water, prior to a subsequent step, e.g., an antibody binding step. In some embodiments, after the blocking step, a sample is not washed prior to a subsequent step, e.g., an antibody binding step.

### Other Preparatory Steps

The methods for detecting a target epitope in a sample disclosed herein may, in some aspects, be performed in conjunction with other sample preparatory steps.

In some embodiments, the method further comprises a sample preparation step. In some embodiments, the sample preparation step comprises preparing a tissue slice from a tissue block. Methods of preparing tissue blocks from specimens are well known in the art. For example, any intact organ or tissue may be cut into small pieces and incubated in various fixatives (e.g., formalin, alcohol, etc.) for varying periods of time until the tissue is “fixed.” The samples may be virtually any intact tissue surgically removed from the body. The samples may be cut into reasonably small piece(s) that fit on the equipment routinely used in histopathology laboratories. The size of the cut pieces typically ranges from a few millimeters to a few centimeters.

In some embodiments, frozen-sections may be prepared by rehydrating 50 mg of frozen “pulverized” tissue at room temperature in phosphate-buffered saline (PBS) in a small plastic capsule; pelleting the particles by centrifugation; resuspending the particles in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

In some embodiments, permanent sections may be prepared by a similar method involving rehydration of the 50 mg sample in a plastic microfuge tube; pelleting; resuspending in 10% formalin for a 4 hour fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

In some embodiments, the sample preparation step comprises a sample fixation step. In some embodiments, the sample is fixed with a fixative. In some embodiments, the fixative is an aldehyde fixative such as formalin (formaldehyde) or glutaraldehyde. Tissue samples fixed using other fixation techniques, such as alcohol immersion, are also suitable. See Battifora and Kopinski, J., Histochem. Cytochem., 34:1095 (1986).

Sample fixation methods are known in the art. See e.g., “Manual of Histological Staining Method of the Armed Forces Institute of Pathology,” 3rd edition (1960) Lee G. Luna, HT (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C. One of skill in the art will appreciate that the choice of the fixative is determined by the purpose for which the tissue is to be histologically stained or otherwise analyzed. One of skill in the art will also appreciate that the length of fixation depends upon the size of the tissue sample and the fixative used. By way of example, neutral buffered formalin, Bouin's, or paraformaldehyde, may be used to fix the tissue sample.

In some embodiments, the sample preparation step comprises embedding a sample in paraffin. In some embodiments, the sample preparation step comprises a deparaffinization step prior to an antibody binging step. In some embodiments, the deparaffinization step comprises deparaffinizing a sample and then rehydrating the sample with water. Deparaffinization are known in the art. In some embodiments, the deparaffinization step comprises washing a sample with an organic solvent to dissolve the paraffin. Such solvents are able to remove paraffin effectively from the tissue sample without adversely affecting the ligands in the tissue. Suitable solvents can be chosen from exemplary solvents, such as benzene, toluene, ethylbenzene, xylenes, and mixtures thereof. A xylene is the preferred solvent for use in the methods of the invention. Solvents alone or in combination in the methods of the invention are preferably of high purity, usually greater than about 99%.

In some embodiments, paraffin is removed by washing with an organic solvent, with vigorous mixing followed by centrifugation. Samples are centrifuged at a speed sufficient to cause the tissue to pellet in the tube, usually at about 10,000 to about 20,000×g. After centrifugation, the organic solvent supernatant is discarded. One of skill in the art of histology will recognize that the volume of organic solvent used and the number of washes necessary will depend on the size of the sample and the amount of paraffin to be removed. The greater the amount of paraffin to be removed, the more washes will be necessary. Typically, the sample will be washed between 1 and about 10 times, and preferably, between about two and about four times. A typical volume of organic solvent is about 500 μL for a 10 μm tissue specimen.

Other methods for deparaffinization known to one of skill in the art may also be used in the method of the disclosure, including direct melting, for example. In additional embodiments, citrus-based aliphatic hydrocarbons (D-Limolene based, for example) may be employed, including other exemplary proprietary formulations used for deparaffinization (e.g., HEMO-DE.RTM. (PMP Medical Industries, Inc., Irving, Tex.); CLEAR-RITE.RTM. (Microm International; Walldorf, Germany); EZ-DEWAX.TM. (BioGenex, San Ramon, Calif.)), for example. EZ-DEWAX.TM. is known to be a de-waxing and rehydration agent.

In some embodiments, the sample is rehydrated after deparaffinization. The preferred method for rehydration is step-wise washing with aqueous lower alcoholic solutions of decreasing concentration. Ethanol is a preferred lower alcohol for rehydration. Other alcohols may also be suitable for use with the invention including methanol, isopropanol and other similar alcohols in the C1-C5 range. The sample is alternatively vigorously mixed with alcoholic solutions and centrifuged. In a preferred embodiment, the concentration range of alcohol is decreased stepwise from about 100% to about 70% in water over about three to five incremental steps, where the change in solution concentration at each step is usually less than about 10% (e.g., the exemplary sequence: 100%, 95%, 90%, 80%, 70%). In some embodiments, deparaffinization and rehydration are carried out simultaneously using a reagent such as EZ-DEWAX.TM. (BioGenex, San Ramon, Calif.), for example.

In some embodiments, the sample preparation step comprises a increasing availability of a target epitope for antibody binding. In some embodiments, increasing availability of a target epitope comprises heat induced epitope retrieval or proteolytic enzyme mediated-epitope retrieval. Citrate buffers, Tris, and EDTA base may be employed as exemplary heat-induced reagents. Pepsin, Proteinase K, Trypsin, Protease, and all of the subtypes may be employed for proteolytic enzyme mediated-epitope retrieval.

### Target Epitopes

The methods disclosed herein may be useful for detecting a target antigen via association with a target epitope thereof. In some embodiments, the target antigen comprises a protein, a carbohydrate, a lipid, and/or a nucleic acid. In some embodiments, the target antigen or a portion thereof (e.g., a target epitope) comprises a protein including a tumor-marker, integrin, cell surface receptor, transmembrane protein, intercellular protein, ion channel, membrane transporter protein, enzyme, antibody, chimeric protein, and glycoprotein. In some embodiments, the target antigen or a portion thereof (e.g., a target epitope) comprises a carbohydrate including a glycoprotein, sugar (e.g., monosaccharide, disaccharide, polysaccharide), and glycocalyx (i.e., the carbohydrate-rich peripheral zone on the outside surface of most eukaryotic cells). In some embodiments, the target antigen or a portion thereof (e.g., a target epitope) comprises a lipid including an oil, fatty acid, glyceride, hormone, steroid (e.g., cholesterol, bile acid), vitamin (e.g., vitamin E), phospholipid, sphingolipid, and lipoprotein.

Numerous target antigens and target epitopes thereof are known in the art. In some embodiments, target antigens and target epitopes thereof include cell surface proteins, e.g., receptors. Exemplary receptors include, but are not limited to: transferrin receptors; LDL receptors; growth factor receptors, such as epidermal growth factor receptor family members (e.g., EGFR, Her2, Her3, and Her4), vascular endothelial growth factor receptors, cytokine receptors, cell adhesion molecules, integrins, selectins, and CD molecules. In some embodiments, target antigens and target epitopes thereof are molecules that are present exclusively or in higher amounts on a malignant cell as compared to a control, e.g., healthy cells or other diseased cells. In some embodiments, target antigens and target epitopes thereof are present in a higher amount than the target antigens and target epitopes thereof in a control, e.g., healthy cells or other diseased cells. In some embodiments, the target antigens and target epitopes thereof are present in a lower amount than the target antigens and target epitopes thereof in a control, e.g., healthy cells or other diseased cells.

In some embodiments, the target analyte and target epitopes thereof are selected from: (a) biomarkers for the diagnosis of undifferentiated neoplasma and/or unknown primary tumors, including epithelial markers (cytokertins and EMA), myoepithelial markers (p63, S100, calponin, SMA, SMMH-1, CK14, maspin), mesenchymal markers (vimentin, SMA, MSA, desmin, MyoD1, Myogenin, NF, S100, P63, CD10, calponin, myoglobin, MDM2, CDK4, FLI-1, CD117, DOG1, CD31, CD34, Factor XIIIa, CD99), melanocytic markers (S100, HMB-45, MART-1, TYROSINASE, MiTF), mesothelial markers (Calretinin, CK5/6, WT1, D2-40, HEME-1, mesothelin, thrombomodulin), neuroendocrine markers (Chromogranin, synaptophysin, CD56, PGP9.5, NSE, insulin, PTH, calcitonin, thyroglobulin, prolactin), germ cell tumor markers (PLAP, OCT4, CD117 or c-kit, SALL4, CD30, alphafetoprotein, beta-hCG, glypican-3, inhibin-alpha, calretinin, EMA, CAM5.2), and hematopoietic markers (CD1a, CD2, CD3, CD5, CD10, CD38, CD21, CD35, CD15, CD30, CD79a, CD43, CD138, CD68, Bcl-2, Bcl-6, cyclin D1, MUMI, S100, MPO); (b) biomarkers for identifying tumor origin, including: calcitonin and CEA for medullary carcinoma of the thyroid; insulin, glucagon and somatostatin for pancreatic endocrine neoplasms; CK20 for merkel cell carcinoma; HMB-4S, MART-1 and SMA for angiomyolipoma; S100, HMB-45, MART-1, SOX10, and vimentin for melanoma; CD117 and DOG-1 for GI and extra-GI stromal tumors; CD5 and p63 for thymic carcinoma; CK20, CDX-2, beta-catenin and villin for colorectal carcinoma; androgen receptor and GCDFP-15 for salivary duct carcinoma; GCDFP-15, ER, PR, mammaglobin for breast carcinoma; TTF1, napsin A and surfactant A for lung adenocarcinoma; TTF1, thyroglobulin, PAX8 for thyroid paoillary and follicular carcinoma; CD1a and S100 for langerhans cell histicytosis; PSA, PSAP and P504S for prostatic adenocarcinoma; CK, EMA, S100 for chordoma; P504S/KIM-1/RCCMa for papillary RCC; RCCMa, KIM-1, PAX8, pVHL for clear cell RCC; MIBI (Ki-67) for hyalinizing trabecular adenoma of the thyroid; OCT4/CD117/PLAP/D2-40 for seminoma; CKs, desmin for desmoplastic small round cell tumor (DPSRCT); glypican-3, Hep Par1 for hepatocellular carcinoma; alpha-fetoprotein/glypican-3/PLAP/SALL4 for yolk sac carcinoma; OCT4/CD30/SOX2/SALL4/PLAP for embryonal carcinoma; DM2, CDK4 for adipose tissue/liposarcoma; myogenin, desmin, myoD1 for rhabdomyosarcoma; SAM, MSA, desmin for leiomyosarcoma/smooth muscle tumor; p16, HPV in situ for cervical and endocervical carcinoma; ER, WT1, PAX8 for ovarian serous carcinoma; CD10, ER for endometrial stromal sarcoma; maspin, VHL for pancreatic ductal adenocarcinoma (PDA); CD2, CD3 for T-cell; CD20, PAX5, CD69a for B-cell; CD43, CD34, CD33, MPO for myeloid cells; CD117, tryptase for mast cells; and CD21, CD35 for follicular dentritic cells; (c) biomarkers for detailed classification within a disease category, selected from CD3, CD20, CD79a, PAX5, CD45rb, CD15, CD30, ALK-1, CD138, CD56, immunoglobulins, HHV8, EMA, TdT, CD34, CD117, and MPO; (d) biomarkers for companion diagnosis, including ER, PR, HER2, EGFR, and CD117 (c-kit); and (e) combinations thereof.

In some embodiments, the target antigen and target epitopes thereof are a tumor marker. In some embodiments, the tumor marker is an antigen that is present in a tumor and that is not present in normal organs, tissues, and/or cells. In some embodiments, the tumor marker is an antigen that is associated with the tumor and is not associated with normal organs, tissues, and/or cells. In some embodiments, the tumor marker is an antigen that is on the cell surface of the tumor and is not on the cell surface of normal organs, tissues, and/or cells. In some embodiments, the tumor marker is an antigen that is more abundant in the tumor than in normal organs, tissues, and/or cells. In some embodiments, the tumor marker is an antigen that is more abundantly associated with the tumor than normal organs, tissues, and/or cells. In some embodiments, the tumor marker is an antigen that is more abundant in malignant cancer cells than in normal cells. In some embodiments, the tumor marker is an antigen that is more abundantly associated with malignant cancer cells than normal cells. In some embodiments, the tumor marker is present at a higher level than in a control. In some embodiments, the tumor marker is present at a higher level than in non-cancerous tissue. In some embodiments, the tumor marker is present at a lower level than in a control. In some embodiments, the tumor marker is present at a lower level than in non-cancerous tissue.

In some embodiments, the tumor marker is a polypeptide. In some embodiments, the tumor marker is a polypeptide, wherein the polypeptide is expressed at higher levels on dividing cells, such as cancerous cells, than on non-dividing cells. For example, Her-2/neu (also known as ErbB-2) is a member of the EGF receptor family and is expressed on the cell surface of tumors associated with breast cancer. Another example is a peptide known as F3 that is a suitable targeting agent for directing a nanoparticle to nucleolin. See Porkka et al., Proc Natl Acad Sci, 99:7444 (2002); and Christian et al., J Cell Biol, 163:871 (2003). It has been shown that targeted particles comprising a nanoparticle and the A10 aptamer (which specifically binds to PSMA) were able to specifically and effectively deliver docetaxel to prostate cancer tumors.

In some embodiments, the target antigen or target epitopes thereof comprises a tumor antigen. “Tumor antigen,” as used herein, is meant an antigenic substance produced in tumor cells, e.g., it triggers an immune response in the host. Normal proteins in the body are not antigenic because of self-tolerance, a process in which self-reacting cytotoxic T lymphocytes (CTLs) and autoantibody-producing B lymphocytes are culled “centrally” in primary lymphatic tissue (BM) and “peripherally” in secondary lymphatic tissue (mostly thymus for T-cells and spleen/lymph nodes for B cells). Thus any protein that is not exposed to the immune system triggers an immune response. This may include normal proteins that are well sequestered from the immune system, proteins that are normally produced in extremely small quantities, proteins that are normally produced only in certain stages of development, or proteins whose structure is modified due to mutation.

In some embodiments, the target antigen (and target epitope thereof) is selected from the group consisting of: CD2, CD19, CD20, CD22, CD27, CD33, CD37, CD38, CD40, CD44, CD47, CD52, CD56, CD70, CD79, and CD137.

In some embodiments, the target antigen (and target epitope thereof) is selected from the group consisting of: 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin αv, Integrin αvβ, KIR, LAG-3, Lewis Y antigen, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and variants thereof. The variants of a target antigen encompass mutants or polymorphisms of the target antigen.

In some embodiments, the target antigen (and target epitope thereof) may be used as a biomarker for identifying tumor origin, including: calcitonin and CEA for medullary carcinoma of the thyroid; insulin, glucagon and somatostatin for pancreatic endocrine neoplasms; CK20 for merkel cell carcinoma; HMB-45, MART-1 and SMA for angiomyolipoma; S100, HMB-45, MART-1, SOX10, and vimentin for melanoma; CD117 and DOG-1 for GI and extra-GI stromal tumors; CD5 and p63 for thymic carcinoma; CK20, CDX-2, beta-catenin and villin for colorectal carcinoma; androgen receptor and GCDFP-15 for salivary duct carcinoma; GCDFP-15, ER, PR, mammaglobin for breast carcinoma; TTF1, napsin A and surfactant A for lung adenocarcinoma; TTF1, thyroglobulin, PAX8 for thyroid paoillary and follicular carcinoma; CD1a and S100 for langerhans cell histicytosis; PSA, PSAP and P504S for prostatic adenocarcinoma; CK, EMA, S100 for chordoma; P504S/KIM-1/RCCMa for papillary RCC; RCCMa, KIM-1, PAX8, pVHL for clear cell RCC; MIB1 (Ki-67) for hyalinizing trabecular adenoma of the thyroid; OCT4/CD117/PLAP/D2-40 for seminoma; CKs, desmin for desmoplastic small round cell tumor (DPSRCT); glypican-3, Hep Par1 for hepatocellular carcinoma; alpha-fetoprotein/glypican-3/PLAP/SALL4 for yolk sac carcinoma; OCT4/CD30/SOX2/SALL4/PLAP for embryonal carcinoma; DM2, CDK4 for adipose tissue/liposarcoma; myogenin, desmin, myoD1 for rhabdomyosarcoma; SAM, MSA, desmin for leiomyosarcoma/smooth muscle tumor; p16, HPV in situ for cervical and endocervical carcinoma; ER, WT1, PAX8 for ovarian serous carcinoma; CD10, ER for endometrial stromal sarcoma; maspin, VHL for pancreatic ductal adenocarcinoma (PDA); CD2, CD3 for T-cell; CD20, PAX5, CD69a for B-cell; CD43, CD34, CD33, MPO for myeloid cells; CD117, tryptase for mast cells; and CD21, CD35 for follicular dentritic cells.

In some embodiments, the target antigen (and target epitope thereof) may be used as a biomarker for identifying more detailed classification within a disease category, such as, CD3, CD20, CD79a, PAX5, CD45rb, CD15, CD30, ALK-1, CD138, CD56, immunoglobulins, HHV8, EMA, TdT, CD34, CD117, and MPO for lymphomas/leukemias.

In some embodiments, the target antigen (and target epitope thereof) may be used as a biomarker for companion diagnosis, including ER, PR, HER2, EGFR, and CD117 (c-kit).

In some embodiments, the target analyte and target epitope thereof is expressed at a low level in the sample. In some embodiments, the copy number of a target analyte is about 1×10³ to 1×10⁴ per cell, such as ROR1 and ROR2. See S. Baskar et al., Unique cell surface expression of receptor tyrosine kinase ROR1 in human B-cell chronic lymphocytic leukemia, Clin Cancer Res 2008:14(2) 396.

### Antibodies

The polymeric-enzyme/antibody conjugates disclosed herein comprise an antibody recognizing a target epitope of a target antigen. In some embodiments, the antibody binds a target epitope disclosed herein.

In some embodiments, the antibody binds an epitope of an epithelial marker (e.g., cytokeratins and EMA), a myoepithelial marker (e.g., p63, S100, calponin, SMA, SMMH-1, CK14, and maspin), a mesenchymal marker (e.g., vimentin, SMA, MSA, desmin, MyoD1, Myogenin, NF, S100, P63, CD10, calponin, myoglobin, MDM2, CDK4, FLI-1, CD117, DOG1, CD31, CD34, Factor XIIIa, and CD99), a melanocytic marker (e.g., S100, HMB-45, MART-1, TYROSINASE, and MiTF), a mesothelial marker (e.g., Calretinin, CK5/6, WT1, D2-40, HBME-1, mesothelin, and thrombomodulin), a neuroendocrine marker (e.g., Chromogranin, synaptophysin, CD56, PGP9.5, NSE, insulin, PTH, calcitonin, thyroglobulin, and prolactin), a germ cell tumor marker (e.g., PLAP, OCT4, CD117 or c-kit, SALL4, CD30, alpha-fetoprotein, beta-hCG, glypican-3, inhibin-alpha, calretinin, EMA, and CAM5.2), a B-Cell marker (e.g., CD79a and PAX5), or a hematopoietic marker (e.g., CD1a, CD2, CD3, CD5, CD10, CD38, CD21, CD35, CD15, CD30, CD79a, CD43, CD138, CD68, Bcl-2, Bcl-6, cyclin D1, MUM1, S100, and MPO).

In some embodiments, the antibody is selected based on its specificity for a target epitope, for example, a target epitope of a target antigen expressed on a target cell, or at a target site, of interest in the tissue sample. A wide variety of tumor-specific or other disease-specific antigens have been identified and antibodies to those antigens have been used or proposed for use in the treatment of such tumors or other diseases. The antibodies that are known in the art can be used in the methods of the present disclosure, in particular for the treatment of the disease with which the target antigen is associated. Examples of target antigens (and their associated diseases) to which an antibody-linker-drug conjugate of the invention can be targeted include: CD2, CD19, CD20, CD22, CD27, CD33, CD37, CD38, CD40, CD44, CD47, CD52, CD56, CD70, CD79, CD137, 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin.alpha.v, Integrin.alpha.v.beta., KIR, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, and VEGFR-3.

In some embodiments, the antibody is an anti-ROR2, anti-Ck8/18, anti-Ki-67, anti-Ck5, anti-Mart-1, anti-S100, or anti-CD45 antibody.

In some embodiments, the antibody is a therapeutic antibody. In some embodiments, the antibody is trastuzumab.

### Polymeric-Enzymes and Polymeric-Enzyme/Antibody Conjugates

The polymeric-enzyme/antibody conjugates disclosed herein comprise a plurality of an enzyme molecule and an antibody recognizing a target epitope. In some embodiments, the polymeric-enzyme/antibody conjugate comprises a plurality of enzyme molecules, wherein the plurality of enzyme molecules comprises the same enzyme type (e.g., all enzyme molecules of an antibody conjugate are horseradish peroxidase). In some embodiments, the polymeric-enzyme/antibody conjugate comprises a plurality of enzyme molecules, wherein the plurality of enzyme molecules comprises different enzyme types.

The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al, Methods for the Preparation of Enzyme-Antibody Conjugates for use in Enzyme Immunoassay, in Methods in Enzym (ed. J. Langone & H. Van Vunakis), Academic press, New York, 73:147-166 (1981). Examples of enzyme-substrate combinations include, for example: (i) Horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB)); (ii) alkaline phosphatase (AP) with para-Nitrophenyl phosphate as chromogenic substrate; and (iii) β-D-galactosidase (β-D-Gal) with a chromogenic substrate (e.g., p-nitrophenyl-β-D-galactosidase), or fluorogenic substrate (e.g., 4-methylumbelliferyl-β-D-galactosidase).

In some embodiments, the enzyme is selected from the group consisting of: β-D-galactosidase, glucose oxidase, horseradish peroxidase, alkaline phosphatase, β-lactamase, glucose-6-phosphate dehydrogenase, urease, uricase, superoxide dismutase, luciferase, pyruvate kinase, lactate dehydrogenase, galactose oxidase, acetylcholine-sterase, enterokinase, tyrosinase, and xanthine oxidase.

In some embodiments, the plurality of enzyme molecules of the polymeric-enzyme are covalently linked. In some embodiments, the plurality of enzyme molecules of the polymeric-enzyme are covalently linked via a crosslinking reagent. In some embodiments, the enzyme comprises a protein component. In some embodiments, the plurality of enzyme molecules of the polymeric-enzyme are covalently linked via a protein component. In some embodiments, the enzyme molecule comprises a polysaccharide component. In some embodiments, the plurality of enzyme molecules of the polymeric-enzyme are covalently linked via a polysaccharide component. In some embodiments, the plurality of enzyme molecules of the polymeric-enzyme are covalently linked via a polysaccharide and a protein component. In some embodiments, the plurality of enzyme molecules of the polymeric-enzyme are non-covalently linked. In some embodiments, the plurality of enzyme molecules comprises a multimeric enzyme. In some embodiments, the plurality of enzyme molecules comprises an enzyme aggregate.

Generally, the polymerization procedure is carried out under conditions which allow for controlled and reproducible formation of the polymeric-enzyme of preselected size. The concentration of the enzyme, the pH of the buffer, the stoichiometry of free functional groups relative to crosslinking reagent, the temperature, and the time of reaction are all important factors in achieving this controllable process.

In some embodiments, the polymeric-enzyme comprises about 5 to about 500 enzyme molecules. In some embodiments, the polymeric-enzyme comprises at least about 5, 10, 15, 20, 25, 50, 75, 100, 150, 200, or 250 enzyme molecules. In some embodiments, the polymeric-enzyme comprises less than about 250, 200, 150, 100, 75, 50, 25, 20, 15, 10, or 5 enzyme molecules.

In some embodiments, the enzyme molecules of the polymeric-enzyme are covalently linked via a crosslinking reagent. In some embodiments, the enzyme molecules of the polymeric-enzyme are covalently linked via a zero-length crosslinking reagent. In some embodiments, the enzyme molecules of the polymeric-enzyme are covalently linked in a linear manner. In some embodiments, the enzyme molecules of the polymeric-enzyme are covalently linked in a branched manner. In some embodiments, the enzyme molecules of the polymeric-enzyme are covalently linked in a mixed linear and branched manner. In some embodiments, the enzyme molecules of the polymeric-enzyme are covalently linked to form a linear structure. In some embodiments, the enzyme molecules of the polymeric-enzyme are covalently linked to form a globular structure.

In some embodiments, the population of polymeric-enzymes comprising a plurality of polymeric-enzymes comprises a size distribution of polymeric-enzymes as characterized by the number of enzyme molecules per polymeric-enzyme. In some embodiments, the population of polymeric-enzymes comprising the plurality of polymeric-enzymes comprises a shape distribution of polymeric-enzymes as characterized by the structure of the polymeric-enzyme.

In some embodiments, the polymeric-enzyme has a molecular weight of about 500 kDa to about 5 mega Daltons (MDa). In some embodiments, the polymeric-enzyme has a molecular weight of at least about 500 kDa. In some embodiments, the polymeric-enzyme has a molecular weight of less than or about 5 MDa. In some embodiments, the polymer-enzyme has a molecular weight of at least about 750 kDa. In some embodiments, the polymer-enzyme has a molecular weight of at least about 1, 2, 3, or 4 MDa.

In some embodiments, the polymeric-enzymes are first formed before being conjugated to antibodies.

Generally, the enzyme is conjugated to an antibody. In some embodiments, more than one enzyme molecule is conjugated to an antibody. In some embodiments, the enzyme molecule is conjugated to more than one antibody. In some embodiments, more than one antibody is conjugated to an enzyme molecule. In some embodiments, more than one enzyme molecule is conjugated to more than one antibody. In some embodiments, more than one antibody is conjugated to more than one enzyme.

By “conjugated” or “attached” or “linked” herein is meant the covalent or non-covalent, as well as direct or indirect, association of a binding agent (such as an antibody) and polymer (such as enzyme polymers) or an enzyme molecule.

Antibody conjugates contemplated in the present invention include those for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase and/or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149; and 4,366,241; each incorporated herein by reference.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al, 1985). The 2- and 8-azido nucleotides have also been used to map nucleotide binding domains of purified proteins (Khatoon et al, 1989; King et al, 1989; and Dholakia et al, 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3a-6a-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors, for example, is achieved using monoclonal antibodies, and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin using reaction conditions that do not alter the antibody combining site are contemplated. Antibody conjugates produced according to this methodology are disclosed to exhibit improved longevity, specificity and sensitivity (U.S. Pat. No. 5,196,066, herein incorporated by reference). Site-specific attachment of effector or reporter molecules, wherein the reporter or effector molecule is conjugated to a carbohydrate residue in the Fc region, have also been disclosed in the literature (O'Shannessy et al., 1987).

Generally, the polymeric-enzyme comprising a plurality of enzyme molecules is conjugated to the antibody. In some embodiments, polymeric-enzyme/antibody conjugates are generated according to methods such as disclosed by U.S. Pat. No. 4,657,853, which is incorporated by reference in its entirety. In some embodiments, the method comprises the sequential steps of: (a) covalently coupling at least two enzyme molecules to produce the polymeric-enzyme; and (b) covalently coupling the polymeric-enzyme to an antibody or fragment thereof.

In some embodiments, the conjugate comprises a Fab, Fab′, F(ab′)2, single domain antibody, T and Abs dimer, Fv, scFv, dsFv, ds-scFv, Fd, linear antibody, minibody, diabody, bispecific antibody fragment, bibody, tribody, sc-diabody, kappa (lamda) body, BiTE, DVD-Ig, SIP, SMIP, DART, or an antibody analogue comprising one or more CDRs.

In some embodiments, the polymeric-enzyme is conjugated to a specific site on the antibody or fragment thereof. In some embodiments, the polymeric-enzyme is conjugated to one or more specific sites on the antibody or fragment thereof. In some embodiments, the polymeric-enzyme is conjugated to a random site on the antibody or fragment thereof. In some embodiments, the polymeric-enzyme is conjugated to one or more random sites on the antibody or fragment thereof. In some embodiments, the polymeric-enzyme is conjugated to the antibody or fragment thereof via an inherent or exogenous chemical characteristic of an amino acid. In some embodiments, the polymeric-enzyme is conjugated to the antibody or fragment thereof via an inherent or exogenous chemical characteristic of an amino acid residue.

In some embodiments, the antibody conjugate comprises one or more polymeric-enzyme. In some embodiments, the antibody conjugate comprises 2, 3, 4, 5, 6, 7, 8 9, 10, 15, or 20 polymeric-enzymes. In some embodiments, the antibody conjugate comprise between 1 and 20 polymeric-enzymes.

In some embodiments, the antibody conjugate comprises between about 6 to about 16, 18, 20, 22, 24, 26, 28, 30, 40, 50, 60, 80, 90, 100, 110, 120, 130, 140, 150, 160, 170, 180, 190, or 200, or more, enzyme molecules per conjugate.

In some embodiments, the antibody conjugate comprises at least between 6-24, between 6-26, between 6-28, between 6-30, between 6-40, between 6-50, between 6-60, between 6-70, between 6-80, between 6-90, or between 6-100 enzyme molecules per conjugate.

In some embodiments, the antibody conjugate comprises at least 6, 8, 10, 12, 14, 16, 18, 20, 30, 40, 50, 60, 70, 80, 90, 100, 110, 120, 130, 140, 150, 160, 170, 180, 190, or 200, but no more than 250, 300, 350, 400, or 500 enzyme molecules per conjugate.

In some embodiments, the antibody conjugate has a molecule weight of between about 400 kDa to about 500 kDa, 600 kDa, 700 kDa, 800 kDa, 900 kDa, 1000 kDa, 2000 kDa, 3000 kDa, 4000 kDa, 5000 kDa, 6000 kDa, 7000 kDa, 8000 kDa, 9000 kDa, or 10000 kDa.

In some embodiments, the antibody conjugate has a molecule weight of between about 470 kDa to about 4.7 megaDa.

In some embodiments, the polymeric-enzyme/antibody conjugate comprises more than one antibody. In some embodiments, the polymeric-enzyme/antibody conjugate comprises a plurality of polymeric-enzymes. In some embodiments, the polymeric-enzyme/antibody conjugate comprises a plurality of polymeric-enzymes, wherein each of the polymeric-enzymes comprise about the same number of the enzyme molecules. In some embodiments, the polymeric-enzyme/antibody conjugate comprises a plurality of polymeric-enzymes, wherein the plurality of polymeric-enzymes exhibit a distribution in the number of enzyme molecules of each polymeric-enzyme. In some embodiments, the polymeric-enzyme/antibody conjugate comprises a plurality of polymeric-enzymes, wherein the plurality of polymeric-enzymes exhibit differences in the shape of the polymeric-enzymes.

In some embodiments, the polymeric-enzyme/antibody conjugate has a ratio (antibody to enzyme) of greater than 1:8. In some embodiments, the polymeric-enzyme/antibody conjugate has a ratio (antibody to enzyme) of greater than 1:6. In some embodiments, the polymeric-enzyme/antibody conjugate has a ratio (antibody to enzyme) of about 1:6, 1:8, 1:15, 1:20, 1:30, 1:40, 1:50, 1:60, 1:75, 1:100, 1:125, 1:150, 1:200.

In some embodiments, the number of polymeric-enzymes conjugated to the polymeric-enzyme/antibody conjugate is adjusted to allow for increased tissue penetration and target analyte detection. In some embodiments, the weight ratio of a polymeric-enzyme/antibody conjugate is adjusted to allow for increased tissue penetration and target analyte detection. In some embodiments, the number of enzymes per polymeric-enzyme conjugated to a polymeric-enzyme/antibody conjugate allows for increased tissue penetration and target analyte detection. In some embodiments, the size of the polymeric-enzyme conjugated to a polymeric-enzyme/antibody conjugate allows for increased tissue penetration and target analyte detection.

In some embodiments, the polymeric-enzyme/antibody conjugate is the conjugate as available from Novodiax, Inc. (Hayward, Calif.), catalogue nos. K29301-1/8, Q31001, Q31002, Q31003, Q31004, Q31005, D28001, D28002, D28003, D28004, D28005, or D28006.

### Enzyme Substrate Compositions

Enzyme substrate compositions allow for detection of one or more target epitopes in a sample using the methods disclosed herein. Each enzyme substrate composition used in the methods disclosed herein may be selected based on the enzyme molecule of the polymeric-enzyme/antibody conjugate used and the constraints of the method (e.g., simultaneous detection in multiplex IHC will require enzyme substrate compositions that can be distinguished from one another).

In some embodiments, the enzyme substrate composition comprises a substrate and a luminescent substrate (such as a chemiluminescent substrate), a chromogenic substrate, a fluorogenic substrate, or a combination thereof. In some embodiments, the enzyme substrate composition comprises a substrate and a luminescent substrate (such as a chemiluminescent substrate). In some embodiments, the enzyme substrate composition comprises a substrate and a chromogenic substrate. In some embodiments, the enzyme substrate composition comprises a substrate and a fluorogenic substrate.

In some embodiments, the first enzyme substrate composition and the second enzyme substrate composition are the same. In some embodiments, the first enzyme substrate composition and the second enzyme substrate composition are different.

In some embodiments, the enzyme substrate composition is incubated with a polymeric-enzyme/antibody conjugate to allow for detection of the target epitope. In some embodiments, the enzyme substrate composition is prepared from a stock solution comprising the enzyme substrate composition. In some embodiments, the solution comprising an enzyme substrate composition, and/or stock solution of the enzyme substrate composition, is free of an impurity. In some embodiments, the solution (e.g., buffer) used to prepare the solution comprising an enzyme substrate composition, and/or stock solution of the enzyme substrate composition, is free of an impurity. In some embodiments, the impurity is an inhibitor of an enzyme molecule.

In some embodiments, the enzyme substrate composition is substantially pure. In some embodiments, the purity of the enzyme substrate composition is at least about any of 80%, 85%, 90%, 95%, 99%, 99.5%, or 99.9% pure.

In some embodiments, the enzyme substrate composition is prepared immediate before an enzyme substrate contact step.

In some embodiments, the enzyme substrate composition comprises one or more of: 3-amino-9-ethylcarbazole (AEC), 3,3′-Diaminobenzidine (DAB), DAB and nickel, AMEC Red, tetramethylbenzidine (TMB), nitro-blue tetrazolium and 5-Bromo-4-chloro-3-indolyl phosphate (NBT/BCIP), naphthol AS-MX phosphate and Fast Red TR (Fast Red), naphthol AS-MX phosphate and Fast Blue BB (Fast Blue), naphthol AS-MX phosphate and new fuchsin (New fuchsin), nitro-blue tetrazolium (NBT), and 5-bromo-4-chloro-3-indolyl-β-D-galactoside (BCIG).

In some embodiments, the enzyme molecules of a polymer-enzyme catalyze more than one substrate type. In some embodiments, the enzyme is horseradish peroxidase and the substrate is AEC (3-amino-9-ethylcarbazole). In some embodiments, the enzyme is horseradish peroxidase and the substrate is DAB (3,3′-diaminobenzidinechromogen). In some embodiments, the enzyme is horseradish peroxidase and the substrate is AMEC Red. In some embodiments, the enzyme is horseradish peroxidase and the substrate is TMB (3,3′,5,5′-tetramethylbenzidine). In some embodiments, the enzyme is alkaline phosphatase and the substrate is Fast Red (Sigma-Aldrich, ST. Louis, Mo.). In some embodiments, the enzyme is alkaline phosphatase and the substrate is Fast Blue (Sigma-Aldrich, ST. Louis, Mo.). In some embodiments, the enzyme is alkaline phosphatase and the substrate is BCIP/NBT (5-bromo-4-chloro-3-indolyl phosphate/nitroblue tetrazolium). Exemplary enzyme/substrate combinations and the respective chromagen color are listed in Table 1.

TABLE 1
Exemplary enzyme substrates.

| Substrate | Enzyme | Chromogen color |
|---|---|---|
| AEC (3-amino-9- | Horseraddish | Red |
| ethylcarbazole) | peroxidase | |
| DAB (3,3′- | Horseraddish | Brown |
| Diaminobenzidine) | peroxidase | |
| DAB + nickel | Horseraddish | Black |
| | peroxidase | |
| TMB | Horseraddish | Blue |
| (Tetramethylbenzidine) | peroxidase | |
| NBT/BCIP (nitro-blue | Alkaline phosphatase | Blue |
| tetrazolium AND 5- | | |
| Bromo-4-chloro-3- | | |
| indolyl phosphate) | | |
| Fast Red (Naphthol | Alkaline phosphatase | Red |
| AS-MX phosphate | | |
| AND Fast Red TR) | | |
| Fast Blue (Naphthol | Alkaline phosphatase | Blue |
| AS-MX phosphate | | |
| AND Fast Blue BB) | | |
| New fuchsin (Naphthol | Alkaline phosphatase | Red |
| AS-MX phosphate | | |
| AND New fuchsin) | | |
| NBT(nitro-blue | Glucose oxidase | Blue |
| tetrazolium) | | |
| BCIG (5-bromo-4- | β-galactosidase | Blue |
| chloro-3-indolyl-β-D- | (BGAL) | |
| galactoside, aka X-gal) | | |

### Sample Type

The methods disclosed herein are useful for detecting a target epitope in a sample. The sample may be any tissue sample, cellular sample, or sub-cellular sample, wherein the sample is suitable for IHC.

In some embodiments, the sample is a formalin-fixed-paraffin-embedded tissue section, a frozen tissue section, a fresh tissue or cell block section, a fresh tissue smear (via core or fine needle biopsy), a fresh tissue via touch imprint, fresh cells from circulating isolation process (e.g., magnetic beads affinity separation, filtration, flow cytometry), fresh cells from touch imprinting, fresh cells from cell cultures, explants, fresh cells isolated from other isolation processes, and fresh micro vesicles, exosomes, or other sub cellular organelles or fragments.

In some embodiments, the sample is a cytology sample. In some embodiments, the cytology sample comprises a clinical smear, a fresh tissue smear, a fresh tissue sample obtained via touch imprint, fresh cells obtained from a circulating isolation process, fresh cells obtained from touch imprinting, fresh cells obtained from cell cultures, explants, fresh cells isolated from other isolation processes, fresh micro-vesicles, exosomes, or other sub cellular organelles or fragments, a body fluid, a body secretion, bronchial alveolar lavage fluid, or cerebrospinal fluid. In some embodiments, the cytology sample is a cellular component sample. In some embodiments, the cellular component sample comprises one or more of the following: micro-vesicles, exosomes, cellular debris, a membrane fragments, and a cellular organelle, or a fragment thereof.

In some embodiments, the sample is derived from an individual. “Individual,” as used herein, is meant any single subject for which therapy is desired, including humans, cattle, dogs, mice, rats, guinea pigs, rabbits, chickens, and insects. Also intended to be included as an individual is any subject involved in a clinical research trial not showing any clinical sign of disease, or an individual involved in an epidemiological study, or an individual used as a control.

“Sample,” as used herein, is meant a collection of similar cells or cellular components obtained from an individual. The source of the sample may be solid tissue, as from a fresh, frozen, and/or preserved organ or tissue sample, or biopsy, or aspirate, or blood or any blood constituents, or bodily fluids, such as cerebral spinal fluid, amniotic fluid, peritoneal fluid, or interstitial fluid, or cells from any time in gestation or development of an individual. The sample may also be primary or cultured cells or cell lines, or culture tissues. The sample may contain compounds which are not naturally intermixed with the sample in nature, such as preservatives, anticoagulants, buffers, fixatives, nutrients, antibiotics, or the like. In some embodiments, the sample is “non-hematologic tissue” (i.e., not blood or bone marrow tissue).

In some embodiments, the sample comprises a cancer cell or cancer cell component. In some embodiments, the sample comprises a cell or cellular component in spatial proximity to a cancer cell. In some embodiments, the sample comprises a cancer cell or cancer cell component and a cell in spatial proximity to a cancer cell. In some embodiments, the sample comprises a cell in close spatial proximity to a cancer cell. In some embodiments, the sample comprises a normal cell or cellular component in close spatial proximity to a cancer cell. In some embodiments, the sample comprises a mixture of cancer cells or cellular components and normal cells in spatial proximity to the cancer cells. In some embodiments, the sample comprises a low percentage of cancer cells. In some embodiments, the sample comprises less than 30%, 20%, 15%, 10%, or 5% cancer cells or cellular components. In some embodiments, the sample comprises between about 5% and about 30% cancer cells or cellular components.

In some embodiments, the sample comprises a tissue section. “Section,” as used herein, is meant a single part or piece of a tissue sample, for example, a thin slice of tissue or cells cut from a tissue sample. It is understood that multiple sections of tissue samples may be taken and subjected to analysis according to the present invention. In some embodiments, the selected portion or section of tissue comprises a homogeneous population of cells. In some embodiments, the selected portion or section of tissue comprises a heterogeneous population of cells. In some embodiments, the selected portion comprises a region of tissue, e.g., the lumen as a non-limiting example. The selected portion can be as small as one cell or two cells, or could represent many thousands of cells, for example. In most cases, the collection of cells is important, and while the invention has been described for use in the detection of cellular components, the method may also be used for detecting non-cellular components of an organism (e.g., soluble components in the blood as a non-limiting example).

In some embodiments, the sample is sourced from the breast, brain, adrenal glands, colon, endometrium, small intestines, stomach, heart, liver, lung, skin, salivary gland, kidney, lung, pancreas, testis, ovary, prostate, uterus, thyroid, or spleen.

In some embodiments, the sample is a tissue section, a clinical smear, or a cultured cell or tissue. In some embodiments, the sample is a section that is more than about 5 μm thick. In some embodiments, the sample is a section that is about 5 μm thick. In some embodiments, the sample is a section that is less than about 5 μm thick. In some embodiments, the sample is a section that is about 1.5 μm thick to about 5.5 μm thick. In some embodiments, the sample is a section that is about 4.5 μm thick to about 7.5 μm thick.

In some embodiments, the tissue is aged. “Aged,” as used herein, is meant tissue that has been stored for a period of time, for example, the period of time that frozen or FFPE are stored. In some embodiments, the sample is a frozen tissue sample. In some embodiments, the sample is paraffin-embedded tissue. In some embodiments, the sample is formalin-fixed paraffin-embedded tissue.

In some embodiments, the sample is fixed in a solution containing an aldehyde. In some embodiments, the sample is fixed in a solution containing formalin. In some embodiments, the sample is paraffin-embedded. In some embodiments, the sample is fixed and embedded in paraffin or the like. In some embodiments, the sample is both formalin-fixed and paraffin-embedded (FFPE).

### Kits and Compositions

The present disclosure provides kits and compositions for the direct IHC staining methods described herein and the applications to multiplex assays, chemically stained samples, cytology samples, and combinations thereof.

In some embodiments, the kit comprises (a) a first polymeric-enzyme/antibody conjugate comprising a plurality of a first enzyme molecule and a first antibody recognizing a first target epitope; and (b) a second polymeric-enzyme/antibody conjugate comprising a plurality of a second enzyme molecule and a second antibody recognizing a second target epitope. In some embodiments, the kit comprises (a) a first polymeric-enzyme/antibody conjugate comprising a plurality of a first enzyme molecule and a first antibody recognizing a first target epitope; (b) a second polymeric-enzyme/antibody conjugate comprising a plurality of a second enzyme molecule and a second antibody recognizing a second target epitope; (c) a first enzyme substrate composition of the first enzyme molecule; and (d) a second enzyme substrate composition of the second enzyme molecule. In some embodiments, the first enzyme molecule and the second enzyme molecule are the same. In some embodiments, the first enzyme molecule and the second enzyme molecule are different. In some embodiments, the first enzyme substrate composition and the second enzyme substrate composition are the same. In some embodiments, the first enzyme substrate composition and the second enzyme substrate composition are different. In some embodiments, the kit further comprises instructions for use according to the methods described herein.

In some embodiments, the kit for visualizing a cellular feature and detecting a first target epitope in a sample comprises: (a) a first polymeric-enzyme/antibody conjugate comprising a plurality of a first enzyme molecule and a first antibody recognizing the first target epitope; and (b) a chemical stain. In some embodiments, the kit for visualizing a cellular feature and detecting a first target epitope in a sample comprises: (a) a first polymeric-enzyme/antibody conjugate comprising a plurality of a first enzyme molecule and a first antibody recognizing a first target epitope; (b) a first enzyme substrate composition of the first enzyme molecule; and (c) a chemical stain. In some embodiments, the kit for visualizing a cellular feature and detecting a first target epitope in a sample further comprises a second polymeric-enzyme/antibody conjugate comprising a plurality of a second enzyme molecule and a second antibody recognizing a second target epitope. In some embodiments, the kit for visualizing a cellular feature and detecting a first target epitope in a sample further comprises a second enzyme substrate composition of the second enzyme molecule. In some embodiments, the first enzyme molecule and the second enzyme molecule are the same. In some embodiments, the first enzyme molecule and the second enzyme molecule are different. In some embodiments, the first enzyme substrate composition and the second enzyme substrate composition are the same. In some embodiments, the first enzyme substrate composition and the second enzyme substrate composition are different. In some embodiments, the kit further comprises instructions for use according to the methods described herein.

In some embodiments, the kit for detecting a target epitope in a cytology sample comprises a first polymeric-enzyme/antibody conjugate comprising a plurality of a first enzyme molecule and a first antibody recognizing the first target epitope. In some embodiments, the kit for detecting a target epitope in a cytology sample comprises (a) a first polymeric-enzyme/antibody conjugate comprising a plurality of a first enzyme molecule and a first antibody recognizing a first target epitope; and (b) a first enzyme substrate composition of the first enzyme molecule. In some embodiments, the kit for detecting a target epitope in a cytology sample further comprises a second polymeric-enzyme/antibody conjugate comprising a plurality of a second enzyme molecule and a second antibody recognizing a second target epitope. In some embodiments, the kit for detecting a target epitope in a cytology sample further comprises a second enzyme substrate composition of the second enzyme molecule. In some embodiments, the first enzyme molecule and the second enzyme molecule are the same. In some embodiments, the first enzyme molecule and the second enzyme molecule are different. In some embodiments, the first enzyme substrate composition and the second enzyme substrate composition are the same. In some embodiments, the first enzyme substrate composition and the second enzyme substrate composition are different. In some embodiments, the kit further comprises instructions for use according to the methods described herein.

The immunodetection reagents of the kit may take any one of a variety of forms, including those detectable labels that are associated with and/or linked to the given primary antibody, preferably as polymeric-enzyme/antibody conjugates.

The kits may further comprise a suitably aliquoted composition of the wild-type and/or mutant protein, polypeptide and/or polypeptide, whether labeled and/or unlabeled, as may be used to prepare the standard curve for a detection assay. The kits may contain antibody-label conjugates either in fully conjugated form, in the form of intermediates, and/or as separate moieties to be conjugated by the user of the kit. The components of the kits may be packaged either in aqueous media and/or in lyophilized form.

The container means of the kits will generally include at least one vial, test tube, flask, bottle, syringe and/or other container means, into which the antibody may be placed, and/or preferably, suitably aliquoted. The kits of the present invention will also typically include a means for containing the antibody, antigen, and/or any other reagent containers in close confinement for commercial sale. Such containers may include injection and/or blow-molded plastic containers into which the desired vials are retained.

The kits of the invention are in suitable packaging. Suitable packaging include, but is not limited to, vials, bottles, jars, flexible packaging (e.g., Mylar or plastic bags), and the like. Kits may optionally provide additional components such as buffers and interpretative information. The present application thus also provides articles of manufacture, which include vials (such as sealed vials), bottles, jars, flexible packaging, and the like.

For example, in one embodiment of the invention, a kit will assess comprehensive panels of molecules (e.g. clinically relevant prognostic and predictive factors in cancer) in broad clinical and research settings.

In some embodiments, the kit will further comprise instructions for use in accordance with any of the methods described herein. The kit may comprise a description on selection of an individual suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

### Methods of Use

The present disclosure provides, in some aspects, methods of using the compositions, kits, and methods described herein. In some embodiments, the method of using the compositions, kits, and methods described herein is for diagnosis. In some embodiments, the method of diagnosis comprises detecting the presence or absence (i.e., a lack of measurable level) of one or more target epitopes in a sample according to the methods described herein. In some embodiments, the methods disclosed herein may be used to select an individual suitable for a treatment agent, such as a therapeutic antibody. In some embodiments, the methods disclosed herein may be used to select a treatment agent, such as a therapeutic antibody, for an individual.

In some embodiments, the method of treating an individual having a disease (such as cancer) comprises detecting one or more epitopes in a sample according to the methods described herein. In some embodiments, the method of treating an individual having a disease further comprises quantifying the level of a target epitope. In some embodiments, the disease is cancer. In some embodiments, the target epitope is a portion of a tumor antigen. In some embodiments, the polymeric-enzyme/antibody conjugate comprises an antibody that binds the tumor antigen.

In some embodiments, methods of using the compositions, kits, and methods described herein comprise stratifying a patient population based on the level of a target antigen, e.g., low, median, and high expression or an expression level at an assigned score. In some embodiments, methods of using the compositions, kits, and methods described herein comprise categorizing a patient or patient population based on the level of a target antigen, e.g., negative expression of a target antigen or presence of a target antigen, including, e.g., low, median, and high expression levels or an expression level at an assigned score.

The methods described herein may allow for high sensitivity detection of a target epitope. For example, in some embodiments, the methods described herein may detect a target epitope, wherein the copy number of the target epitope is about 1×10⁴ or less per cell, such as 1×10³ or less per cell. In some embodiments, methods of using the compositions, kits, and methods described herein may allow for diagnosing an individual with a disease, such as cancer, or a sub-type of the disease, wherein the individual has not been previously diagnosed with the disease and/or the sub-type of the disease.

In some embodiments, there is provided a method of selecting (including identifying) an individual having a disease (such as cancer) characterized by an abnormal level of a target epitope for treatment with an agent that targets the target epitope, comprising: detecting the presence and/or level of the target epitope according to the methods disclosed herein.

In some embodiments, the methods disclosed herein are useful for: (i) assessing whether an individual having a disease will likely respond to treatment; (ii) selecting (including identifying) an individual having a disease likely to respond to treatment; (iii) adjusting therapy treatment of an individual having a disease receiving an effective amount of an agent that targets a target analyte; (iv) predicting a measurable reduction in tumor size or evidence of disease or disease progression, complete response, partial response, stable disease, increase or elongation of progression free survival, or increase or elongation of overall survival; (v) predicting/prolonging progression-free survival; (vi) reducing AEs and SAEs; (vii) predicting/improving quality of life; and (viii) determining a tissue distribution of a target epitope.

The methods for detecting one or more target epitopes in a sample described herein may also be useful for determining any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits. In some embodiments, the level of target analyte as determined using a polymeric-enzyme/antibody conjugate can also be useful for aiding assessment in any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits.

The presence of or level of target epitope may be measured before, after, and/or during a treatment. In some embodiments, the values obtained can be used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; or (g) predicting likelihood of clinical benefits.

In some embodiments, the individual is human. In some embodiments, the individual is a female. In some embodiments, the individual is a male. In some embodiments, the individual is under about 65 years old. In some embodiments, the individual is at least about 65 years old, at least about 70 years old, or at least about 75 years old. In some embodiments, the individual has one or more symptoms of chronic stress, including physical and psychological stress associated with the cancer, such as anxiety, depression, headache, pain, fatigue, insomnia, anorexia, nausea, malnutrition, or any combination thereof. In some embodiments, the individual has an advanced stage of cancer, such as any of T2, T3, T4, N1, N2, N3 or M1 stage of cancer based on the TNM staging system. In some embodiments, the individual has a high tumor burden, such as a large tumor size and/or a large number of cancer cells in the tumor bed. In some embodiments, the individual has palpable lymph nodes, or has cancer cells spread to nearby lymph nodes. In some embodiments, the individual has distant tumor metastases.

In some embodiments, the disease is cancer. In some embodiments, the cancer is selected from the group consisting of lung cancer, uterine cancer, kidney cancer, ovarian cancer, breast cancer, endometrial cancer, head and neck cancer, pancreatic cancer, and melanoma. In some embodiments, the cancer is selected from the group consisting of breast cancer, lung cancer, and pancreatic cancer. In some embodiments, the cancer is triple negative breast cancer (TNBC). In some embodiments, the cancer is non-small cell lung cancer (NSCLC). In some embodiments, the cancer is pancreatic ductal adenocarcinoma (PDAC). In some embodiments, the cancer is selected from the group consisting of adrenocortical cancer, bile duct cancer, bladder cancer, breast cancer, cervical cancer, colon cancer, endometroid cancer, esophageal cancer, glioblasoma, head and neck cancer, kidney chromophobe cancer, kidney clear cell carcinoma, kidney papillary cell carcinoma, liver cancer, lower grade glioma, lung adenocarcinoma, lung squamous cell carcinoma, melanoma, mesothelioma, ocular melanomas, ovarian cancer, pancreatic cancer, pheochromocytoma and paraganglioma, prostate cancer, sarcoma, stomach cancer, testicular cancer, thyroid cancer, and uterine carcinosarcoma.

In some embodiments, the cancer is a solid epithelial tumor or a sarcoma. In some embodiments, the cancer is selected from a group consisting of adrenocortical carcinoma, Kaposi sarcoma, anal cancer, gastrointestinal carcinoid tumor, basal cell carcinoma, bile duct cancer, bladder cancer (such as bladder transitional cell carcinoma, bladder squamous cell carcinoma, and bladder adenocarcinoma), bone cancer (such as Ewing Sarcoma, osteosarcoma, chondrosarcoma, and malignant fibrous histiocytoma), breast cancer (such as ductal carcinoma, lobular carcinoma, fibroadenoma), bronchial tumor, carcinoma of unknown primary, cervical cancer, chordoma, colon cancer, rectal cancer, endometrial cancer, esophageal cancer (including esophageal squamous cell carcinoma and esophageal adenocarcinoma), intraocular melanoma, ovarian cancer (such as ovarian epithelial cancer, Fallopian tube cancer, and peritoneal cancer), gallbladder cancer, gastric cancer, head and neck cancer (such as hypopharyngeal cancer, laryngeal cancer, lip and oral cavity cancer, metastatic squamous neck cancer with occult primary treatment, nasopharyngeal cancer, oropharyngeal cancer, paranasal sinus and nasal cavity cancer, salivary gland cancer, and oral complications of chemotherapy and head/neck radiation), heart tumor (such as rhabdomyoma, myxoma, fibroma, fibrosarcoma, and angiosarcoma), hepatocellular (liver) cancer, kidney cancer (such as renal cell cancer, transitional cell cancer of the renal pelvis and ureter, and Wilms tumor), lung cancer (such as non-small cell lung cancer, and small cell lung cancer), skin cancer (such as basal cell carcinoma, squamous cell carcinoma, neuroendocrine carcinoma of the skin, melanoma, and Merkel cell carcinoma), pancreatic cancer, pheochromocytoma, parathyroid cancer, penile cancer, pituitary tumor, prostate cancer, uterine sarcoma (such as leiomyosarcoma and endometrial stromal sarcoma), small intestine cancer (such as small intestine adenocarcinoma and small intestine sarcoma, and gastrointestinal stromal tumor), soft tissue sarcoma (such as adult soft tissue sarcoma, and childhood soft tissue sarcoma), thyroid cancer (such as papillary, follicular, medullary and anaplastic thyroid cancer), urethral cancer (including urethral transitional cell carcinoma, urethral squamous cell carcinoma, and urethral adenocarcinoma), vaginal cancer (such as vaginal squamous cell carcinoma and vaginal adenocarcinoma), a cancer of a sweat gland, and vulvar cancer. In some embodiments, the cancer comprises infiltrating cells.

In some embodiments, the cancer is at an advanced stage (such as stage III or stage IV). In some embodiments, the cancer is metastatic cancer.

In some embodiments, the treatment method is a first-line therapy.

The classification or ranking of the target epitope level (e.g., high or low) as determined according to the methods described herein may be determined relative to a statistical distribution of control levels. In some embodiments, the classification or ranking is relative to a control sample, such as a normal tissue. In some embodiment, the level of a target eptiope is classified or ranked relative to a statistical distribution of control levels. In some embodiments, the level of a target epitope is classified or ranked relative to the level from a control sample obtained from the individual.

Control samples can be obtained using the same sources and methods as non-control samples. In some embodiments, the control sample is obtained from a different individual (for example an individual not having cancer, an individual having a benign or less advanced form of a disease corresponding to the cancer, and/or an individual sharing similar ethnic, age, and gender identity). In some embodiments, when the sample is a tumor tissue sample, the control sample may be a non-cancerous sample from the same individual. In some embodiments, multiple control samples (for example from different individuals) are used to determine a range of levels of target analytes in a particular tissue, organ, or cell population.

In some embodiments, bioinformatics methods are used for the determination and classification of the levels of the target analyte.

In some embodiments, the target epitiope level is determined according to the methods described herein, for example, by direct multiplex immunohistochemistry. For example, the criteria for low or high levels can be made based on the number of positive staining cells and/or the intensity of the staining, for example by using an antibody that specifically recognizes the target analyte. In some embodiments, the level is low if less than about 1%, 5%, 10%, 15%, 20%, 25%, 30%, 35%, 40%, 45%, or 50% cells have positive staining. In some embodiments, the level is low if the staining is 1%, 5%, 10%, 15%, 20%, 25%, 30%, 35%, 40%, 45%, or 50% less intense than a positive control staining. In some embodiments, the level is high if more than about 40%, 45%, 50%, 55%, 60%, 65%, 70%, 75%, 80%, 85%, or 90%, cells have positive staining.

In some embodiments, the level is high if the staining of a polymeric-enzyme/antibody conjugate is as intense as positive control staining. In some embodiments, the level is high if the staining of a polymeric-enzyme/antibody conjugate is 80%, 85%, or 90% as intense as positive control staining of a polymeric-enzyme/antibody conjugate.

In some embodiments, strong staining, moderate staining, and low staining are calibrated levels of staining of a polymeric-enzyme/antibody conjugate, wherein a range is established and the intensity of staining is binned within the range. In some embodiments, strong staining is staining of a polymeric-enzyme/antibody conjugate above the 75th percentile of the intensity range, moderate staining is staining of a polymeric-enzyme/antibody conjugate from the 25th to the 75th percentile of the intensity range, and low staining is staining is staining of a polymeric-enzyme/antibody conjugate below the 25th percentile of the intensity range. In some aspects one skilled in the art, and familiar with a particular staining technique, adjusts the bin size and defines the staining categories.

In some embodiments, the assessment and scoring of the target analyte level as in a sample, patient, etc., as determined by a polymeric-enzyme/antibody conjugate is performed by one or more experienced clinicians, i.e., those who are experienced with target analyte expression and target analyte staining patterns using a polymeric-enzyme/antibody conjugate. For example, in some embodiments, the clinician(s) is blinded to clinical characteristics and outcome for the samples, patients, etc. being assessed and scored.

In some embodiments, the methods described herein are performed in a clinic. In some embodiments, the methods described herein are performed outside of a clinic. In some embodiments, the methods described herein are performed at a diagnostic lab.

Those skilled in the art will recognize that several embodiments are possible within the scope and spirit of the present invention. The following examples illustrate the invention but, of course, should not be construed as in any way limiting its scope.

### EXAMPLES

### Example 1

This example demonstrates duplex IHC staining of frozen skin tissue to distinguish cancerous tissue from healthy tissue.

Direct IHC staining using an anti-CK5 HRP polymeric-enzyme/antibody conjugate (polyHRP-CK5) and an anti-CK8/18 HRP polymeric-enzyme/antibody conjugate (polyHRP-CK8/18) was performed on intraoperatively obtained frozen skin tissue sections. CK5 is a cytokeratin that has demonstrated utility when directed at basal cell carcinoma and squamous cell carcinoma for determining surgical margins during skin cancer surgery. However, when only targeting CK5 during IHC staining, the morphology of exocrine gland cells, such as sweat glands or sebaceous glands, may make it difficult to distinguish healthy tissue from cancerous tissue. To more accurately determine margins in surgical treatment of skin cancers, duplex staining using polyHRP-CK5 and polyHRP-CK8/18 was performed on frozen surgical margins from a patient with squamous carcinoma. After applying an enzyme substrate composition comprising the chromogen DAB (to generate a red brown color), HRP was inactivated by 30% H₂O₂ for 30 minutes. PolyHRP-CK5 was then applied and the sample was contacted with a second enzyme substrate composition comprising a second chromogen that generated a greenish color.

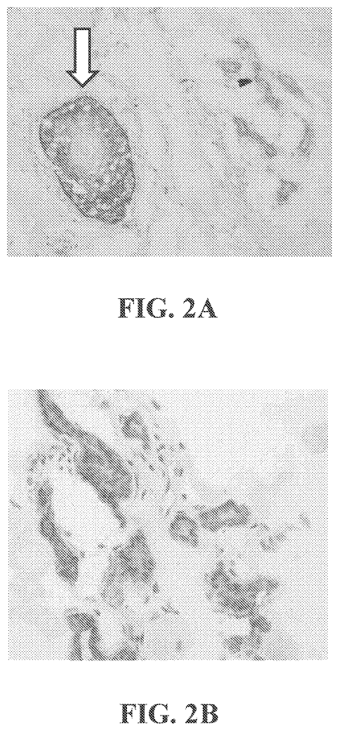

Using polyHRP-CK5 and polyHRP-CK8/18 duplex staining, the sweat gland was stained with polyHRP-CK8/18 in red brown with normal morphology (FIG. 2A, arrow). In proximity on the right of the sweat gland, the cancerous tissue was stained by both polyHRP-CK5 in green and by polyHRP-CK8/18 in red brown and was clearly distinguishable from the sweat gland. FIG. 2B provides a zoomed in image of the cancerous tissue which was stained by both polyHRP-CK5 in green and by polyHRP-CK8/18 in red brown.

### Example 2

This example demonstrates triplex staining of formalin-fixed paraffin-embedded (FFPE) breast cancer tissue.

Direct IHC staining using an anti-HER2 HRP polymeric-enzyme/antibody conjugate (polyHRP-Herceptin), an anti-CK8/18 HRP polymeric-enzyme/antibody conjugate (polyHRP-CK8/18), and an anti-Ki-67 HRP polymeric-enzyme/antibody conjugate (polyHRP-Ki-67) was performed.

Breast cancer tissue was first stained with polyHRP-Herceptin using DAB as a chromogen to generate brown color in the membrane of carcinoma cells. Following inactivation of HRP by 30% H₂O₂ for 30 minutes, the tissue was subsequently stained by polyHRP-CK8/18 and a yellow chromogen to stain the cytoplasma. After a second inactivation step to inactivate HRP, the tissue was further stained with polyHRP-ki67 and AEC as a chromogen which generate red purple color in the nucleus.

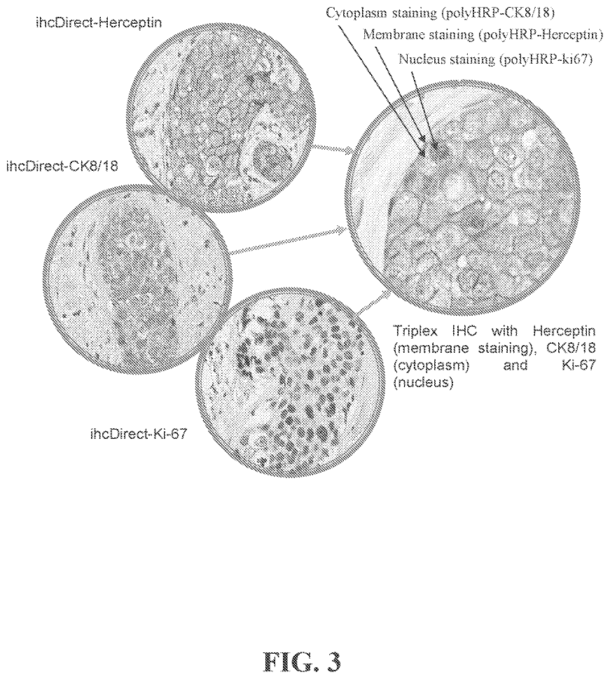

Using the triplex staining method, an improved image of the stained tissue was obtained, wherein polyHRP-Herceptin stained the cell membranes, polyHRP-CK8/18 stained the cytoplasm, and polyHRP-Ki-67 stained the nucleus (FIG. 3).

### Example 3

This example demonstrates a direct ICC staining of a cell smear from a fine needle aspiration biopsy of the lung. The fine needle aspiration biopsy sample was previously stained with hematoxylin and eosin and had been archived for years.

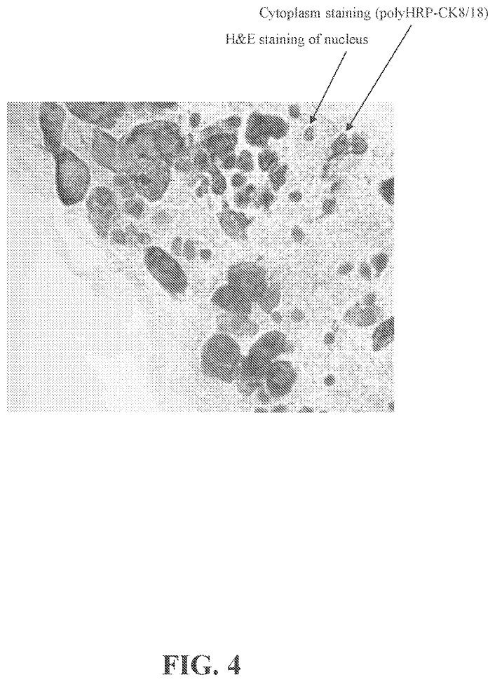

Direct ICC staining using an anti-CK8/18 HRP polymeric-enzyme/antibody conjugate (polyHRP-CK8/18) was performed. The polyHRP-CK8/18 stained the cytoplasm of intact epithelium cells of the cytology sample previously stained with the chemical stain hematoxylin and eosin (FIG. 4).

What is claimed is:

1. A method for detecting a plurality of target epitopes in a sample, the method comprising:
(a) a first antibody binding step comprising contacting the sample with a first polymeric-enzyme/antibody conjugate comprising a plurality of a first enzyme molecule and a first antibody recognizing a first target epitope in a condition suitable for forming a first complex comprising the first target epitope and the first polymeric-enzyme/antibody conjugate;
(b) a second antibody binding step comprising contacting the sample with a second polymeric-enzyme/antibody conjugate comprising a plurality of a second enzyme molecule and a second antibody recognizing a second target epitope in a condition suitable for forming a second complex comprising the second target epitope and the second polymeric-enzyme/antibody conjugate;
(c) a first antibody removal step comprising substantially removing from the sample the first polymeric-enzyme/antibody conjugate that does not form the first complex;
(d) a second antibody removal step comprising substantially removing from the sample the second polymeric-enzyme/antibody conjugate that does not form the second complex;
(e) a first enzyme substrate contact step comprising contacting the sample with a first enzyme substrate composition for the first enzyme molecule; and
(f) a second enzyme substrate contact step comprising contacting the sample with a second enzyme substrate composition for the second enzyme molecule,
thereby allowing for detection of the plurality of target epitopes in the sample.

2. The method of claim 1, wherein the first enzyme molecule and the second enzyme molecule are different.

3. The method of claim 2, wherein the first antibody binding step and the second antibody binding step are performed simultaneously.

4. The method of claim 2 or 3, wherein the first antibody removal step and the second antibody removal step are performed simultaneously.

5. The method of any one of claims 2-4, wherein the first enzyme substrate contact step and the second enzyme substrate contact step are performed simultaneously.

6. The method of any one of claims 2-4, wherein the first enzyme substrate contact step is performed prior to the second enzyme substrate contact step.

7. The method of any one of claims 2-4, wherein the first enzyme substrate contact step is performed after the second enzyme substrate contact step.

8. The method of claim 2, wherein the first antibody binding step is performed prior to the second antibody binding step.

9. The method of claim 8, wherein the first antibody removal step and the first enzyme substrate contact step are performed prior to the second antibody binding step.

10. The method of claim 9, further comprising a first antibody stripping step comprising dissociating the first antibody from the first target epitope, wherein the first antibody stripping step is performed prior to the second antibody binding step.

11. The method of claim 9, further comprising a first enzyme inactivation step comprising inactivating the first enzyme molecule, wherein the first enzyme inactivation step is performed prior to the second antibody binding step.

12. The method of claim 2, wherein the first antibody binding step is performed after the second antibody binding step.

13. The method of claim 12, wherein the second antibody removal step and the second enzyme substrate contact step are performed prior to the first antibody binding step.

14. The method of claim 13, further comprising a second antibody stripping step comprising dissociating the second antibody from the second target epitope, wherein the second antibody stripping step is performed prior to the first antibody binding step.

15. The method of claim 13, further comprising a second enzyme inactivation step comprising inactivating the second enzyme molecule, wherein the second enzyme inactivation step is performed prior to the first antibody binding step.

16. The method of claim 1, wherein the first enzyme molecule and the second enzyme molecule are the same.

17. The method of claim 16, wherein the first antibody binding step is performed prior to the second antibody binding step.

18. The method of claim 17, wherein the first antibody removal step and the first enzyme substrate contact step are performed prior to the second antibody binding step.

19. The method of claim 18, further comprising a first antibody stripping step comprising dissociating the first antibody from the first target epitope, wherein the first antibody stripping step is performed prior to the second antibody binding step.

20. The method of claim 18, further comprising a first enzyme inactivation step comprising inactivating the first enzyme molecule, wherein the first enzyme inactivation step is performed prior to the second antibody binding step.

21. The method of claim 16, wherein the first antibody binding step is performed after the second antibody binding step.

22. The method of claim 21, wherein the second antibody removal step and the second enzyme substrate contact step are performed prior to the first antibody binding step.

23. The method of claim 22, further comprising a second antibody stripping step comprising dissociating the second antibody from the second target epitope, wherein the second antibody stripping step is performed prior to the first antibody binding step.

24. The method of claim 22, further comprising a second enzyme inactivation step comprising inactivating the second enzyme molecule, wherein the second enzyme inactivation step is performed prior to the first antibody binding step.

25. A method for visualizing a cellular feature and detecting a first target epitope in a sample, the method comprising:
(a) a chemical staining step comprising contacting the sample with a chemical stain; and
(b) a first antibody binding step comprising contacting the sample with a first polymeric-enzyme/antibody conjugate comprising a plurality of a first enzyme molecule and a first antibody recognizing a first target epitope in a condition suitable for forming a first complex comprising the first target epitope and the first polymeric-enzyme/antibody conjugate,
thereby allowing for visualization of the cellular feature and detection of the first target epitope.

26. The method of claim 25, wherein the chemical stain is hematoxylin and eosin (H&E) stain, papanicolaou (PAP) stain, giemsa stain, alican blue stain, mucicarmine stain, periodic acid-Schiff (PAS) stain, Masson's trichrome stain, Jone's stain, Hall's stain, iron-based stain, and Luxol fast blue stain.

27. A method for detecting a first target epitope in a sample, the method comprising:
a first antibody binding step comprising contacting the sample with a first polymeric-enzyme/antibody conjugate comprising a plurality of a first enzyme molecule and a first antibody recognizing a first target epitope in a condition suitable for forming a first complex comprising the first target epitope and the first polymeric-enzyme/antibody conjugate,
wherein the sample is a cytology sample,
thereby allowing for detection of the first target epitope.

28. The method of claim 27, wherein the sample is a clinical smear sample, a core needle biopsy sample, a fine needle biopsy sample, or a touch imprint sample.

29. The method of any one of claims 25-28, wherein the method further comprises detecting a second target epitope in the sample, the method comprising:
a second antibody binding step comprising contacting the sample with a second polymeric-enzyme/antibody conjugate comprising a plurality of a second enzyme molecule and a second antibody recognizing the second target epitope in a condition suitable for forming a second complex comprising the second target epitope and the second polymeric-enzyme/antibody conjugate,
thereby allowing for detection of the second target epitope.

30. The method of claim 29, further comprising:
a first enzyme substrate contact step comprising contacting the sample with a first enzyme substrate composition for the first enzyme molecule; and
a second enzyme substrate contact step comprising contacting the sample with a second enzyme substrate composition for the second enzyme molecule.

31. The method of any one of claims 1-24 and 30, wherein the first enzyme substrate composition comprises a substrate and a chromogenic substrate, a chemiluminescent substrate, a fluorogenic substrate, or a combination thereof.

32. The method of any one of claims 1-24, 30, and 31, wherein the second enzyme substrate composition comprises a substrate and a chromogenic substrate, a chemiluminescent substrate, a fluorogenic substrate, or a combination thereof.

33. The method of any one of claims 1-24 and 30-32, further comprising a first detection step comprising detecting a first substrate reporter generated from the first enzyme substrate composition by the first enzyme molecule.

34. The method of any one of claims 1-24 and 30-33, further comprising a second detection step comprising detecting a second substrate reporter generated from the second enzyme substrate composition by the second enzyme molecule.

35. The method of any one of claims 1-34, wherein the first antibody binding step or the second antibody binding step comprises incubating the first polymeric-enzyme/antibody conjugate or the second polymeric-enzyme/antibody conjugate, respectively, with the sample.

36. The method of any one of claims 1-35, wherein the first binding step or the second binding step is performed for an incubation period of about 3 minutes to about 30 minutes.

37. The method of any one of claims 1-36, wherein the first binding step or the second binding step is performed at an incubation temperature of between about 15° C. to about 37° C.

38. The method of any one of claims 1-37, wherein the first antibody removal step or the second antibody removal step comprises contacting the sample with a wash buffer.

39. The method of any one of claims 1-38, wherein the first antibody removal step or the second antibody removal step is performed for an incubation period of about 1 minute and about 60 minutes.

40. The method of any one of claims 1-39, wherein the first antibody removal step or the second antibody removal step is performed 1-10 times.

41. The method of any one of claims 1-40, wherein the first antibody removal step or the second antibody removal step is performed at an incubation temperature of between about 15° C. and about 50° C.

42. The method of any one of claims 1-41, wherein the first enzyme substrate composition or the second enzyme substrate composition is a solution.

43. The method of any one of claims 1-42, wherein the first enzyme substrate contact step or the second enzyme substrate contact step is performed for an incubation period of about 1 minute and about 60 minutes.

44. The method of any one of claims 1-43, wherein the first enzyme substrate contact step or the second enzyme substrate contact step is performed at an incubation temperature of between about 15° C. and about 50° C.

45. The method of any one of claims 1-44, further comprising a blocking step prior to the first antibody binding step and/or the second antibody binding step, wherein the blocking step comprises contacting the tissue with a blocking agent.

46. The method of claim 45, wherein the blocking agent comprises skim milk, BSA, cold fish skin gelatin, casein, or an animal serum.

47. The method of any one of claims 1-46, wherein the sample is a frozen sample.

48. The method of any one of claims 1-47, wherein the sample is fixed in a fixing solution comprising an aldehyde.

49. The method of claim 48, wherein the fixing solution comprises formalin.

50. The method of any one of claims 1-49, wherein the sample is paraffin-embedded.

51. The method of claim 50, wherein the sample is a formalin-fixed-paraffin-embedded sample.

52. The method of any one of claims 1-26 and 29-51, wherein the sample is a tissue section.

53. The method of claim 52, wherein the tissue section is about 1.5 μm to about 5.5 μm thick.

54. The method of any one of claims 1-51, wherein the sample is a cell block section.

55. The method of claim 54, wherein the cell block section is about 1.5 μm to about 5.5 μm thick.

56. The method of any one of claims 1-46, wherein the sample is a fresh tissue sample.

57. The method of any one of claims 1-56, wherein the first enzyme molecule or the second enzyme molecule is selected from the group consisting of: beta-D-galactosidase, glucose oxidase, horseradish peroxidase, alkaline phosphatase, beta-lactamase, glucose-6-phosphate dehydrogenase, urease, uricase, superoxide dismutase, luciferase, pyruvate kinase, lactate dehydrogenase, galactose oxidase, acetylcholine-sterase, enterokinase, tyrosinase, and xanthine oxidase.

58. The method of any one of claims 1-57, wherein the polymeric-enzyme/antibody conjugate comprises at least 6 enzyme molecules per polymeric-enzyme/antibody conjugate.

59. The method of claim 58, wherein the polymeric-enzyme/antibody conjugate comprises between about 6 and about 80 enzyme molecules per polymeric-enzyme/antibody conjugate.

60. The method of any one of claims 1-59, wherein the enzyme molecules of the polymeric-enzyme are covalently linked.

61. The method of any one of claims 1-60, wherein the polymeric-enzyme has a molecular weight of about 500 kDa to about 5 MDa.

62. The method of any one of claims 1-61, wherein the polymeric-enzyme/antibody conjugate has an antibody to enzyme ratio of greater than about 1:6.

63. The method of any one of claims 1-62, wherein the antibody is a therapeutic antibody.

64. A kit comprising:
(a) a first polymeric-enzyme/antibody conjugate comprising a plurality of a first enzyme molecule and a first antibody recognizing a first target epitope;
(b) a second polymeric-enzyme/antibody conjugate comprising a plurality of a second enzyme molecule and a second antibody recognizing a second target epitope,
wherein the first target epitope and the second target epitope are different.

65. A kit comprising:
(a) a first polymeric-enzyme/antibody conjugate comprising a plurality of a first enzyme molecule and a first antibody recognizing a first target epitope;
(b) a chemical stain.

66. The kit of claim 64 or 65, further comprising instructions for use according to the methods of any one of claims 1-64.
